(19) United States
(12) Patent Application Publication
(10) Pub. No.: US 2024/0366405 A1
(43) Pub. Date: Nov. 7, 2024

(54) INTELLIGENT IMPLANTS AND ASSOCIATED COMPONENTS AND ASSEMBLY TOOLS

(71) Applicant: CANARY MEDICAL INC., Vancouver (CA)

(72) Inventors: Michael Boothby, San Diego, CA (US); Kevin Gemmell, San Diego, CA (US); Jeffrey M. Gross, Carlsbad, CA (US); John Ray, Indian Creek, IL (US); Peter J. Schiller, Shakopee, MN (US); Matthew Sturtevant, San Diego, CA (US)

(21) Appl. No.: 18/568,658

(22) PCT Filed: Jun. 14, 2022
(86) PCT No.: PCT/US2022/033417

§ 371 Date: Dec. 8, 2023

Related U.S. Application Data
(60) Provisional application No. 63/217,709, filed on Jul. 1, 2021.
(60) Provisional application No. 63/217,704, filed on Jul. 1, 2021.
(60) Provisional application No. 63/217,696, filed on Jul. 1, 2021.
(60) Provisional application No. 63/210,445, filed on Jun. 14, 2021.

(51) Int. Cl. A61F 2/48; A61F 2/30; A61F 2/36; A61F 2/38; A61F 2/40

(52) U.S. Cl. CPC A61F 2/488; A61F 2/36; A61F 2/3859; A61F 2/389; A61F 2/4014; A61F 2002/3067

(57) ABSTRACT

An intelligent implant includes an implantable reporting processor (IRP) and a component of a prothesis system having a receptacle. The IRP includes an antenna, an electronics assembly including a sensor, a hermetically sealed chamber containing the electronics assembly, a casing configured to house at least a portion of the electronics assembly, and a cover configured to house the antenna. The hermetically sealed chamber contains a gas. The casing and the cover abut one another to form an antenna chamber that houses the antenna and a filler. The receptacle of the component is configured to receive a portion of the IRP and to mechanically couple with the implantable reporting processor. The component of the prothesis system may be one of a tibial component or a femoral component of a knee prosthesis system, a humeral component of a shoulder prosthesis system, and a femoral component of a hip prosthesis system.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### TECHNICAL FIELD

The present disclosure relates generally to intelligent implants associated with implantable systems such as orthopedic (e.g., joint replacement systems), and more particularly, to intelligent implants with implantable reporting processors that sample, record, and transmit information related to the placement and integrity of an implanted system, and the health of the patient in which the system is implanted, as well as features of intelligent implants including space-efficient circuit assemblies therefor, enhanced transmitting antenna configurations therefor, and tools for assembling intelligent implants

### BACKGROUND

Orthopedic replacement systems, such as knee arthroplasty systems, shoulder arthroplasty systems, hip arthroplasty systems, and spinal implant systems may be configured to replace the entirety of a knee, shoulder, or hip joint, or to replace a part of knee, shoulder, or hip. Systems intended to replace the entirety of a knee, shoulder, or hip joint are referred to as total joint replacement systems or total joint arthroplasty (TJA), while those intended to replace a part of a joint are referred to as partial joint replacement systems. In either case, these joint replacement systems include implant structures or components.

With reference to FIG. 1A, a total knee arthroplasty (TKA) typically consists of a femoral component, a tibial component, a tibial insert, a tibial stem extension and a patella component. The patella component, which is implanted in front of the joint, is not shown. Collectively, these five implant structures or components may be referred to as any one of an implantable medical device, a knee prosthetic system, or total knee implant (TKI). Each of these five components may also be individually referred to as an implantable medical device. In either case, these components are designed to work together as a functional unit, to replace and provide the function of a natural knee joint.

With reference to FIG. 1B, a standard total shoulder arthroplasty (TSA) typically consists of a humeral stem component, a humeral stem adapter, a humeral head, a humeral head adapter (not shown), and a glenoid cap component. Collectively, these four implant structures or components may be referred to as any one of an implantable medical device, a shoulder prosthetic system, or total shoulder implant (TSI). Each of these four components may also be individually referred to as an implantable medical device. In either case, these components are designed to work together as a functional unit, to replace and provide the function of a natural shoulder joint.

With reference to FIG. 1C, a total hip arthroplasty (THA) typically consists of a femoral stem component, a femoral head component, a head liner component, and an acetabular cap component. Collectively, these four implant structures or components may be referred to as any one of an implantable medical device, a hip prosthetic system, or total hip implant (THI). Each of these four components may also be individually referred to as an implantable medical device. In either case, these components are designed to work together as a functional unit, to replace and provide the function of a natural hip joint.

Current commercial TJA systems have a long history of clinical use with implant duration regularly exceeding 10 years and with some reports supporting an 87% survivorship at 25 years. Clinicians currently monitor the progress of TJA patients post implant using a series of physical exams at 2-3 weeks, 6-8 weeks, 3 months, 6 months, 12 months, and yearly thereafter.

After the TJA has been implanted, and the patient begins to walk with the knee or hip prosthesis and move his arms or shoulder prosthesis, problems may occur and are sometimes hard to identify. Clinical exams are often limited in their ability to detect failure of the prosthesis; therefore, additional monitoring is often required such as CT scans, MRI scans or even nuclear scans. Given the continuum of care requirements over the lifetime of the implant, patients are encouraged to visit their clinician annually to review their health condition, monitor other joints, and assess the TJA implant's function. While the current standard of care affords the clinician and the healthcare system the ability to assess a patient's TJA function during the 90-day episode of care, the measurements are often subjective and lack temporal resolution to delineate small changes in functionality that could be a pre-cursor to larger mobility issues. The long-term (>1 year) follow up of TJA patients also poses a problem in that patients do not consistently see their clinicians annually. Rather, they often seek additional consultation only when there is pain or other symptoms.

Currently, there is no mechanism for reliably detecting misplacement, instability, or misalignment in many medical implants (such as in the TJA) without clinical visits and the hands and visual observations of an experienced health care provider. Even then, early identification of subclinical problems or conditions is either difficult or impossible since they are often too subtle to be detected on physical exam or demonstratable by radiographic studies. Furthermore, if detection were possible, corrective actions would be hampered by the fact that the specific amount of movement and/or degree of improper alignment cannot be accurately measured or quantified, making targeted, successful intervention unlikely. Existing external monitoring devices do not provide the fidelity required to detect instability since these devices are separated from the TJA by skin, muscle, and fat—each of which masks the mechanical signatures of instability and introduce anomalies such as flexure, tissue-borne acoustic noise, inconsistent sensor placement on the surface, and inconsistent location of the external sensor relative to the TJA.

In addition, a patient may experience a number of complications post-procedure. Such complications include neurological symptoms, pain, malfunction (blockage, loosening, etc.) and/or wear of the implant, movement or breakage of the implant, inflammation and/or infection. While some of these problems can be addressed with pharmaceutical products and/or further surgery, they are difficult to predict and prevent; often early identification of complications and side effects, although desirable, is difficult or impossible.

The present disclosure is directed to various mechanical aspects of intelligent implants with implantable reporting processors that sample, record, and transmit information related to the placement and integrity of an implanted TJA, and the health of the patient in which the TJA is implanted.

### SUMMARY

Briefly stated, the present disclosure relates to an implantable reporting processor (IRP) of an intelligent implant. The IRP includes a casing and a cover. The casing is configured to house at least a portion of an electronics assembly, and includes a distal facing wall and an externally threaded portion positioned distally from the distal facing wall. The externally threaded portion is separated from the distal facing wall by an externally smooth surface. The cover is configured to house an antenna electrically coupled to the electronics assembly, and includes an internally threaded portion spaced apart from a proximal edge of the cover by an internally smooth surface. The externally threaded portion of the casing is configured to engage with the internally threaded portion of the cover.

The present disclosure also relates to an IRP that includes an antenna, an electronics assembly including a sensor, a casing configured to house the electronics assembly, and a cover. The cover is configured to house the antenna, and is sufficiently strong that it can withstand anatomical fatigue loading resulting from forces exerted on the cover after the implantable reporting processor has been implanted in a bone of a subject and the subject performs normal daily activity.

The present disclosure also relates to an impaction sleeve for coupling an IRP to a component of a joint replacement system. The impaction sleeve is configured to transfer impaction forces from an impaction tool to the IRP. The impaction sleeve includes an elongate portion comprising a channel extending longitudinally through the elongate portion and a distal head at an end of the elongate portion, wherein the distal head comprises a larger width than the elongate portion. The channel is configured to receive at least a portion of an IRP through a proximal opening. The distal head includes an end face and a peripheral surface. The end face of the distal head is configured to receive the impaction forces from the impaction tool.

The present disclosure also relates to an IRP that includes a casing, a battery, an antenna configured to transmit data, and an electronics assembly at least partially enclosed by the casing. The electronics assembly includes a flexible circuit assembly, a liner, and a sleeve. The circuit assembly is coupled to the battery and to the antenna, and is configured to generate data related to the implantable reporting processor. The flexible circuit assembly includes a first portion and a second portion that can be folded to overlap each other. The liner includes a first section configured to receive the first portion of the flexible circuit assembly and a second section configured to receive the second portion of the flexible circuit assembly. When the flexible circuit assembly is folded, the liner encloses the flexible circuit assembly. The sleeve is configured to enclose the liner, and includes a distal rim configured to abut a proximally facing surface of the casing.

The present disclosure also relates to an IRP that includes an antenna, an electronics assembly including a sensor, a hermetically sealed chamber containing the electronics assembly, a casing configured to house at least a portion of the electronics assembly, and a cover configured to house the antenna. The hermetically sealed chamber contains a gas.

The present disclosure also relates to an IRP that includes an antenna, an electronics assembly including a sensor, a casing configured to house at least a portion of the electronics assembly at least partially within a hermetically sealed chamber, and a cover configured to house the antenna. The casing and the cover abut one another to form an antenna chamber that houses the antenna and a filler.

The present disclosure also relates to an intelligent implant that includes an IRP as described in any of the preceding paragraphs, and a component of a prothesis system having a receptacle. The receptacle is configured to receive a portion of the IRP and to mechanically couple with the implantable reporting processor. The component of the prothesis system may be one of a tibial component or a femoral component of a knee prosthesis system, a humeral component of a shoulder prosthesis system, and a femoral component of a hip prosthesis system.

The present disclosure also relates to an intelligent implant that includes a component of a prosthesis system and an IRP coupled to the component. The IRP has a casing that includes a shoulder and a proximal end, and a proximal portion located between the shoulder and the proximal end. The proximal portion comprises a coupling region having an indent that is annularly symmetrical around a perimeter of the coupling region.

This Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1A is an illustration of a conventional implantable medical device in the form of a knee prosthetic system, or total knee implant (TKI).

FIG. 1B is an illustration of a conventional implantable medical device in the form of a shoulder prosthetic system, or total shoulder implant (TSI).

FIG. 1C is an illustration of a conventional implantable medical device in the form of a hip prosthetic system, or total hip implant (THI).

FIGS. 2A and 2B are illustrations of an intelligent implant in the form of a tibial component of a knee prosthesis including a tibial plate and a long-extension implantable reporting processor extending from a tibial stem.

FIGS. 3A, 3B, and 3C are illustrations of an intelligent implant in the form of a tibial component of a knee prosthesis including a tibial plate and a short-extension implantable reporting processor extending from a tibial stem.

FIGS. 4A and 4B are illustrations of an intelligent implant in the form of a tibial component of a knee prosthesis including a tibial plate and an implantable reporting processor integrated with a tibial stem.

FIGS. 5A and 5B are illustrations of an intelligent implant in the form of a humeral component of a shoulder prosthesis including an implantable reporting processor integrated with a humeral stem.

FIGS. 6A and 6B are illustration of an intelligent implant in the form of a femoral component of a hip prosthesis including an implantable reporting processor integrated with a femoral stem.

FIG. 7 is illustration of an intelligent implant in the form of a femoral component of a knee prosthesis.

FIGS. 8A, 8B, and 8C are illustrations of an intelligent implant in the form of a tibial component of a knee prosthesis.

FIGS. 9A, 9B, and 9C are illustrations of an intelligent implant in the form of a tibial component of a knee prosthesis.

FIG. 10A is a perspective view of an embodiment of an implantable reporting processor.

FIG. 10B is an exploded view of the implantable reporting processor of FIG. 10A.

FIG. 11A is an exploded view of an embodiment of a subassembly of the implantable reporting processor of FIG. 10A.

FIG. 11B is a perspective view of an assembled subassembly of FIG. 11A.

FIG. 12A is an exploded view of another embodiment of a subassembly of the implantable reporting processor of FIG. 10A.

FIG. 12B is a partially exploded view of the subassembly of FIG. 12A.

FIG. 12C is a perspective view of an assembled subassembly of FIG. 12A.

FIG. 12D is a perspective view of the subassembly of FIG. 12A with an antenna.

FIG. 13 is a side view of a casing of the implantable reporting processor of FIG. 10A.

FIG. 14A is a perspective view of the casing shown in FIG. 10A, and the subassembly of FIG. 11B.

FIG. 14B is a perspective view of the subassembly of FIG. 11B enclosed by the casing of FIG. 10A.

FIG. 14C is a perspective view of the subassembly of FIG. 11B enclosed by the casing of FIG. 10A with an antenna attached.

FIG. 14D is a perspective view of the casing with subassembly and antenna shown in FIG. 14C with a cover attached to the casing to enclose the antenna.

FIGS. 15A and 15B are partial cross-sectional views of the cover of FIG. 14D coupled to the casing.

FIG. 16 is an illustration of the intelligent implant (humeral component with integrated implantable reporting processor) of FIGS. 5A and 5B.

FIGS. 17A and 17B are cross-section illustrations of configurations of the intelligent implant of FIG. 16.

FIG. 18 is an exploded illustration of the implantable reporting processor of the intelligent implant of FIG. 16.

FIGS. 19A, 19B, 19C, 19D and 19E are a series of illustrations depicting an assembling of the intelligent implant of FIG. 16.

FIG. 20 is an illustration of the intelligent implant (femoral component with integrated implantable reporting processor) of FIGS. 6A and 6B.

FIGS. 21A and 21B are perspective views of a folded circuit assembly enclosed by liners.

FIG. 22A is a bottom view of the circuit assembly coupled to a configuration of liners.

FIG. 22B is a top view of the circuit assembly coupled to the liners of FIG. 22A.

FIGS. 22C, 22D, 22E and 22F are perspective views of the printed circuit assembly coupled to the liners of FIG. 22A.

FIG. 23A is a perspective view of the circuit assembly of FIGS. 21A and 21B in a flat configuration.

FIGS. 23B and 23C are illustrations of the circuit assembly of the electronics assembly of FIGS. 13C.

FIG. 23D is a perspective view of a folded the circuit assembly of FIG. 23A folded.

FIG. 24A is a perspective view of the circuit assembly enclosed by liners of FIGS. 12A and 12B.

FIG. 24B is a cross-sectional view of the circuit assembly enclosed by liners of FIG. 24A.

FIGS. 24C and 24D are exploded views of the circuit assembly enclosed by liners of FIG. 24A.

FIG. 24E is a perspective view of the liners of FIGS. 12A and 12B.

FIG. 25 is a block diagram of an implantable reporting processor (IRP).

FIGS. 26A, 26B, and 26C are different views of the use of a tool to couple two portions of the tibial component of FIGS. 9A, 9B, and 9C.

FIGS. 27A, 27B, and 27C are different views of the tool of FIGS. 26A, 26B, and 26C.

FIGS. 28A, 28B, 28C, 28D, 28E, and 28F are different views of an embodiment of a tool.

FIG. 29 is a flowchart of a method of assembling a first component and a second component of an intelligent implant.

### DETAILED DESCRIPTION

The present disclosure may be understood more readily by reference to the following detailed description of embodiments of the disclosure and the examples of implantable medical devices with implantable reporting processors. The following description, along with the accompanying drawings, sets forth certain specific details in order to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure, including but not limited to the communication systems and networks, have not been shown or described in order to avoid unnecessarily obscuring descriptions of the embodiments.

The present disclosure refers to TJA (total joint arthroplasty) which term includes reference to the surgery and associated implantable medical devices such as a TJA prosthesis. Features of methods, devices and systems of the present disclosure may be illustrated herein by reference to a specific TJA prosthesis, however, the disclosure should be understood to apply to any one or more TJA prosthesis, including a TKA (total knee arthroplasty) prosthesis, such as a TKI (total knee implant) which may also be referred to as a TKA system; a TSA (total shoulder arthroplasty) prosthesis, such as a TSI (total shoulder implant) which may also be referred to as a TSI system; and a THA (total hip arthroplasty) prosthesis, such as a THI (total hip implant) which may also be referred to as a THA system.

An “implantable medical device” as used in the present disclosure, is an implantable or implanted medical device that desirably replaces or functionally supplements a subject's natural body part. Examples of implantable medical devices include orthopedic implants such as knee, hip, and shoulder implants, as well as spinal implant systems (e.g., a spinal fusion implant such as a spinal interbody cage, rod or plate, or a spinal non-fusion implant such as an artificial disc or expandable rod).

As used herein, the term “intelligent implant” refers to an implantable medical device with an implantable reporting processor, and is interchangeably referred to a “smart device.” When the intelligent implant makes kinematic measurements, it may be referred to as a “kinematic implantable device.” In describing embodiments of the present disclosure, reference may be made to a kinematic implantable device, however it should be understood that this is exemplary only of the intelligent medical devices which may be employed in the devices, methods, systems etc. of the present disclosure.

In one embodiment, the intelligent implant is an implanted or implantable medical device having an implantable reporting processor arranged to perform the functions as described herein. The intelligent implant may perform one or more of the following exemplary actions in order to characterize the post-implantation status of the intelligent implant: identifying the intelligent implant or a portion of the intelligent implant, e.g., by recognizing one or more unique identification codes for the intelligent implant or a portion of the intelligent implant; detecting, sensing and/or measuring parameters, which may collectively be referred to as monitoring parameters, in order to collect operational, kinematic, or other data about the intelligent implant or a portion of the intelligent implant and wherein such data may optionally be collected as a function of time; storing the collected data within the intelligent implant or a portion of the intelligent implant; and communicating the collected data and/or the stored data by a wireless means from the intelligent implant or a portion of the intelligent implant to an external computing device. The external computing device may have or otherwise have access to at least one data storage location such as found on a personal computer, a base station, a computer network, a cloud-based storage system, or another computing device that has access to such storage.

Non-limiting and non-exhaustive list of embodiments of intelligent implants include components of a total knee arthroplasty (TKA) system, a total hip arthroplasty (THA) system, a total shoulder arthroplasty (TSA) system, an intramedullary rod for arm or leg breakage repair, a scoliosis rod, a dynamic hip screw, spinal implants (e.g., a spinal fusion implant such as a spinal interbody cage, rod or plate, or a spinal non-fusion implant such as an artificial disc or expandable rod), an annuloplasty ring, a heart valve, an intravascular stent, a vascular graft, and a vascular stent graft.

“Kinematic data,” as used herein, individually or collectively includes some or all data associated with a particular kinematic implantable device and available for communication outside of the particular kinematic implantable device. For example, kinematic data may include raw data from one or more sensors of a kinematic implantable device, wherein the one or more sensors include such as gyroscopes, accelerometers, pedometers, strain gauges, and the like that produce data associated with motion, force, tension, velocity, or other mechanical forces. Kinematic data may also include processed data from one or more sensors, status data, operational data, control data, fault data, time data, scheduled data, event data, log data, and the like associated with the particular kinematic implantable device. In some cases, high resolution kinematic data includes kinematic data from one, many, or all of the sensors of the kinematic implantable device that is collected in higher quantities, resolution, from more sensors, more frequently, or the like.

In one embodiment, kinematics refers to the measurement of the positions, angles, velocities, and accelerations of body segments and joints during motion. Body segments are considered to be rigid bodies for the purposes of describing the motion of the body. They include the foot, shank (leg), thigh, pelvis, thorax, hand, forearm, upper-arm, and head. Joints between adjacent segments include the ankle (talocrural plus subtalar joints), knee, hip, wrist, elbow, and shoulder. Position describes the location of a body segment or joint in space, measured in terms of distance, e.g., in meters. A related measurement called displacement refers to the position with respect to a starting position. In two dimensions, the position is given in Cartesian co-ordinates, with horizontal followed by vertical position. In one embodiment, a kinematic implant or intelligent kinematic implants obtains kinematic data, and optionally only obtains only kinematic data.

“Sensor” refers to a device that can be utilized to do one or more of detect, measure and/or monitor one or more different aspects of a body tissue (anatomy, physiology, metabolism, and/or function) and/or one or more aspects of the orthopedic device or implant. Representative examples of sensors suitable for use within the present disclosure include, for example, fluid pressure sensors, fluid volume sensors, contact sensors, position sensors, pulse pressure sensors, blood volume sensors, blood flow sensors, chemistry sensors (e.g., for blood and/or other fluids), metabolic sensors (e.g., for blood and/or other fluids), accelerometers, mechanical stress sensors and temperature sensors. Within certain embodiments the sensor can be a wireless sensor, or, within other embodiments, a sensor connected to a wireless microprocessor. Within further embodiments one or more (including all) of the sensors can have a Unique Sensor Identification number (“USI”) which specifically identifies the sensor. In certain embodiments, the sensor is a device that can be utilized to measure in a quantitative manner, one or more different aspects of a body tissue (anatomy, physiology, metabolism, and/or function) and/or one or more aspects of the orthopedic device or implant. In certain embodiments, the sensor is an accelerometer that can be utilized to measure in a quantitative manner, one or more different aspects of a body tissue (e.g., function) and/or one or more aspects of the orthopedic device or implant (e.g., alignment in the patient).

A wide variety of sensors (also referred to as Microelectromechanical Systems or “MEMS,” or Nanoelectromechanical Systems or “NEMS,” and BioMEMS or BioNEMS, see generally https://en.wikipedia.org/wiki/MEMS) can be utilized within the present disclosure. Representative patents and patent applications include U.S. Pat. Nos. 7,383,071, 7,450,332; 7,463,997, 7,924,267 and 8,634,928, and U.S. Publication Nos. 2010/0285082, and 2013/0215979. Representative publications include “Introduction to BioMEMS” by Albert Foch, CRC Press, 2013; “From MEMS to Bio-MEMS and Bio-NEMS: Manufacturing Techniques and Applications by Marc J. Madou, CRC Press 2011; “Bio-MEMS: Science and Engineering Perspectives, by Simona Badilescu, CRC Press 2011; “Fundamentals of BioMEMS and Medical Microdevices” by Steven S. Saliterman, SPIE-The International Society of Optical Engineering, 2006; “Bio-MEMS: Technologies and Applications”, edited by Wanjun Wang and Steven A. Soper, CRC Press, 2012; and “Inertial MEMS: Principles and Practice” by Volker Kempe, Cambridge University Press, 2011; Polla, D. L., et al., “Microdevices in Medicine,” Ann. Rev. Biomed. Eng. 2000, 02:551-576; Yun, K. S., et al., “A Surface-Tension Driven Micropump for Low-voltage and Low-Power Operations,” J. Microelectromechanical Sys., 11:5, October 2002, 454-461; Yeh, R., et al., “Single Mask, Large Force, and Large Displacement Electrostatic Linear Inchworm Motors,” J. Microelectromechanical Sys., 11:4, August 2002, 330-336; and Loh, N. C., et al., “Sub-10 cm3 Interferometric Accelerometer with Nano-g Resolution,” J. Microelectromechanical Sys., 11:3, June 2002, 182-187; all of the above of which are incorporated by reference in their entirety.

### Intelligent Implants

The present disclosure provides intelligent implants, e.g., an implantable medical device with an implantable reporting processor (IRP). When the intelligent implant is included in a component of an implant system that replaces a joint, the intelligent implant can monitor displacement or movement of the component or implant system. The intelligent implant can also provide kinematic data that can be used to assess the mobility and health of the patient in which the system is implanted.

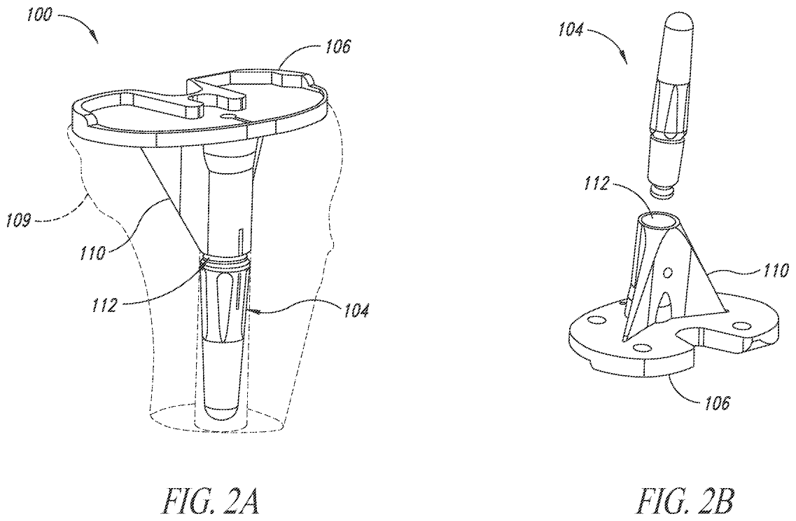

With reference to FIGS. 2A, 2B, 3A, 3B, 3C, 4A, 4B, and 7 an intelligent implant 100, 160, 300, 400 may be part of a component of a knee implant system. In FIGS. 2A, 2B, 3A, 3B, 3C, 4A, and 4B, the intelligent implant 100, 300, 400 corresponds to a tibial component of a knee replacement system for a TKA and includes a tibial plate 106, 306, 406 and an implantable reporting processor (IRP) 104, 304, 404. The tibial plate 106, 306, 406 is configured to physically attach to an upper surface of a tibia 109. A tibial stem 110, 310, 410 or tibial keel extends from the tibial plate 106, 306, 406. The tibial stem 110, 310, 410 includes a receptacle 112, 312, 412 configured to receive a portion of the implantable reporting processor 104, 304, 404.

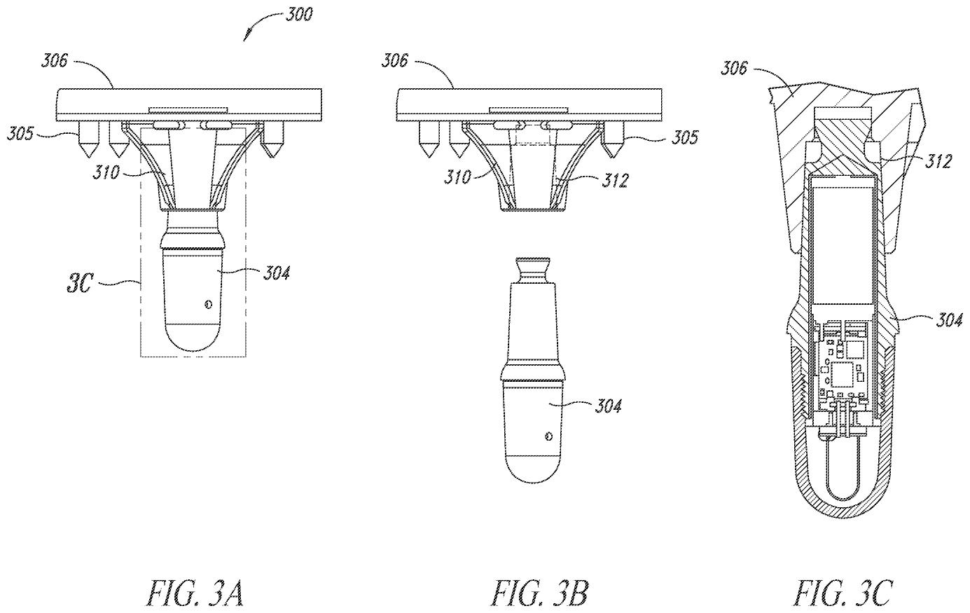

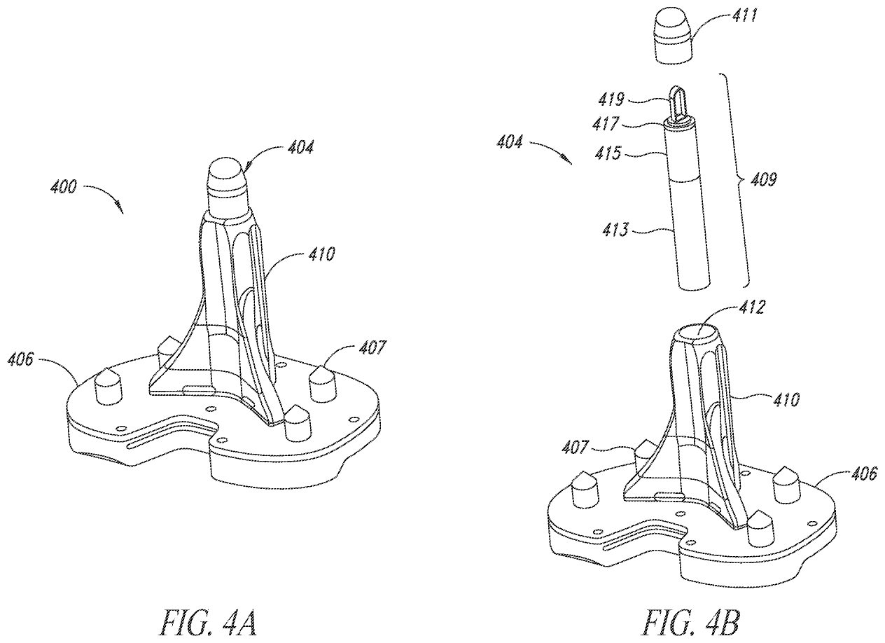

In the embodiment of FIGS. 2A and 2B, the tibial plate 106 is adhered or glued to the upper surface of the tibia using a biocompatible cement to establish a physical attachment between the tibial component 100 and the tibia. In the embodiments of FIGS. 3A, 3B, 3C, 4A, and 4B, a portion of the tibial plate 306, 406 is configured to adhere to the upper surface of the tibia in the absence of cement. To this end, the portion of the tibial plate 306, 406 may be the lower surface facing the tibia and may include a layer of porous ingrowth material into which boney tissue grows to secure the tibial plate in place. The porous layer may be formed for example, by cobalt-chromium sintered beads, titanium fiber metal mesh, cancellous-structured titanium, and titanium plasma spray. A number of projections 305, 405 or pegs may extend from the lower surface. These projections 305, 405 are configured to be forced into the upper surface of the tibia and establish an initial fixation or physical attachment between the tibial component 300, 400 and the tibia. The embodiments of FIGS. 3A, 3B, 3C, 4, and 5 are referred to as cementless tibial components. Patient selection for cementless tibial components tend to be younger patients with healthier bone (not osteoporotic). The fundamental concept for cementless tibial component design is to provide “wings” for rotational stability but minimize the amount of bone that is being removed, hence the tibial stem of these components tend to be narrow. Minimal bone removal is one of the preferred aspects of cementless. Cemented tibias require more bone removal to create a cement mantel around the implant.

In the embodiment of FIGS. 2A, 2B, 3A, 3B, and 3C, the implantable reporting processor 104, 304 is a component assembly manufactured separate from the tibial plate 106, 306 and is mechanically coupled to the tibial stem 110, 310 of the tibial plate to form the tibial component 100, 300. To this end, a portion of the implantable reporting processor 104, 304 is inserted into the receptacle 112, 312 of the tibial stem 110, 310 and fixed in place therein by application of a force that couples respective mechanical features of the implantable reporting processor and the tibial plate 106, 306. In the embodiments of FIGS. 4A and 4B, the implantable reporting processor 404 is integrated into the tibial stem 410 of the tibial plate 406 during assembly. To this end, a subassembly 409 of the implantable reporting processor 104 is inserted into the receptacle 412 of the tibial stem 410 and a cover 411 of the implantable reporting processor is coupled to the subassembly 409 and the tibial stem. The subassembly 409 includes a battery 413, an electronics assembly 415, an antenna feedthrough 417, and an antenna 419.

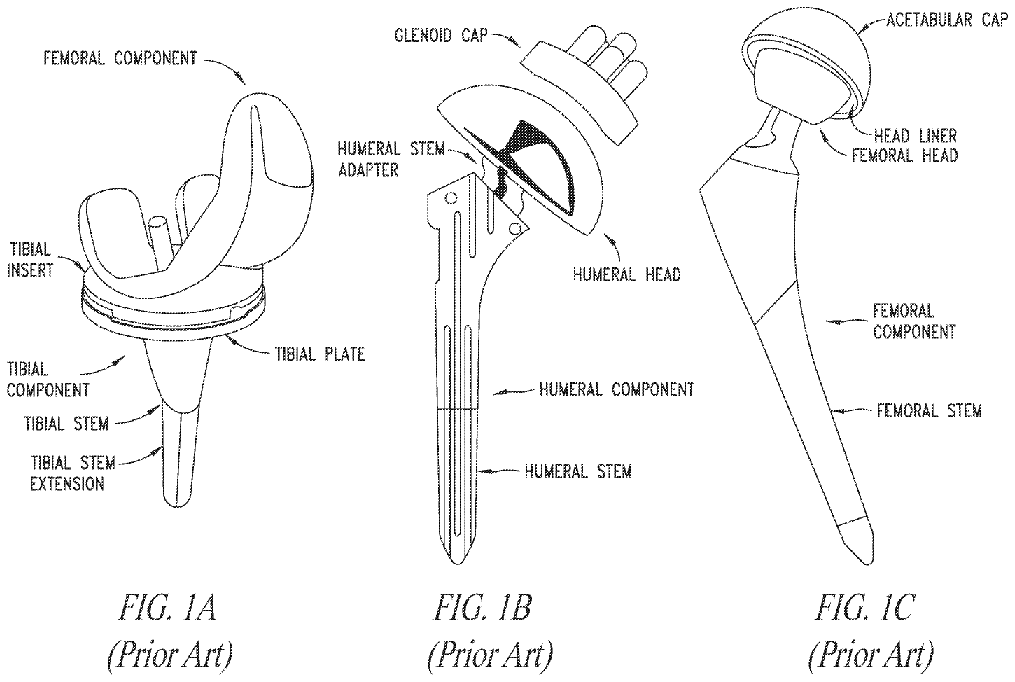

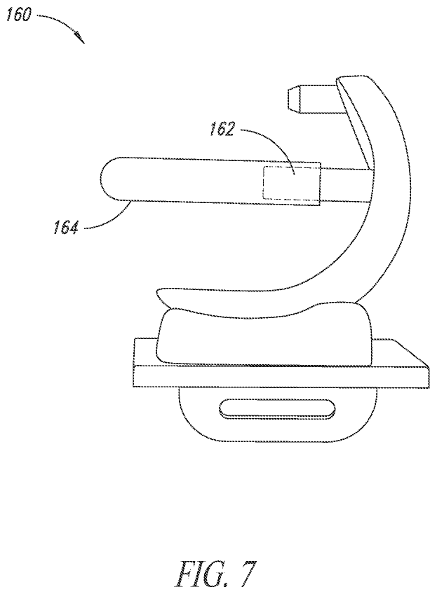

In FIG. 7, the intelligent implant 160 corresponds to a femoral component of a knee implant system. In the embodiment of FIG. 7, the femoral component is a unicompartmental femoral component used in partial knee arthroplasty (PKA). The implantable reporting processor 164 may be configured to physically attach to an extension 162 of the femoral component. In other embodiments, the femoral component may be of the type shown in FIG. 1A that is used for a TKA.

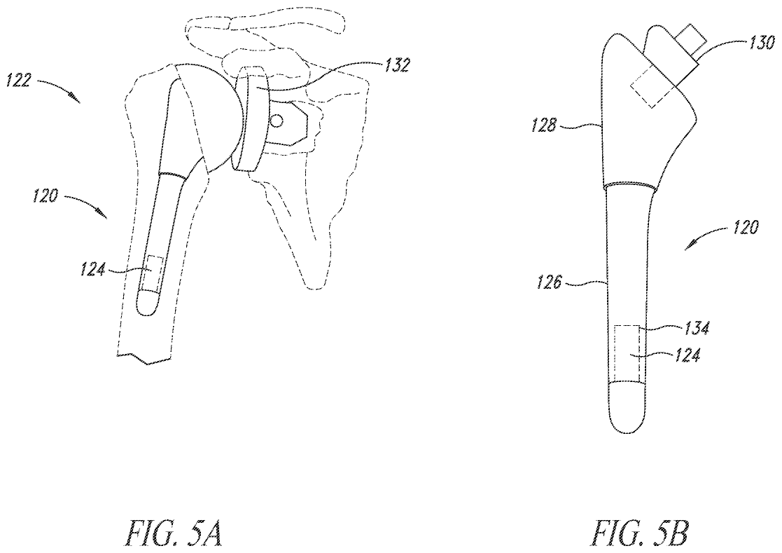

With reference to FIGS. 5A and 5B, an intelligent implant 120 may be part of a shoulder implant system 122. In this embodiment, the intelligent implant 120 corresponds to a humeral component of a shoulder replacement system for a TSA and includes an implantable reporting processor (IRP) 124. The humeral component 120 includes a humeral stem 126, a humeral body 128, and a humeral head adapter 130. The humeral head adapter 130 is configured to physically attach to glenoid cap 132. The humeral stem 126 includes a receptacle 134 configured to receive the implantable reporting processor 124.

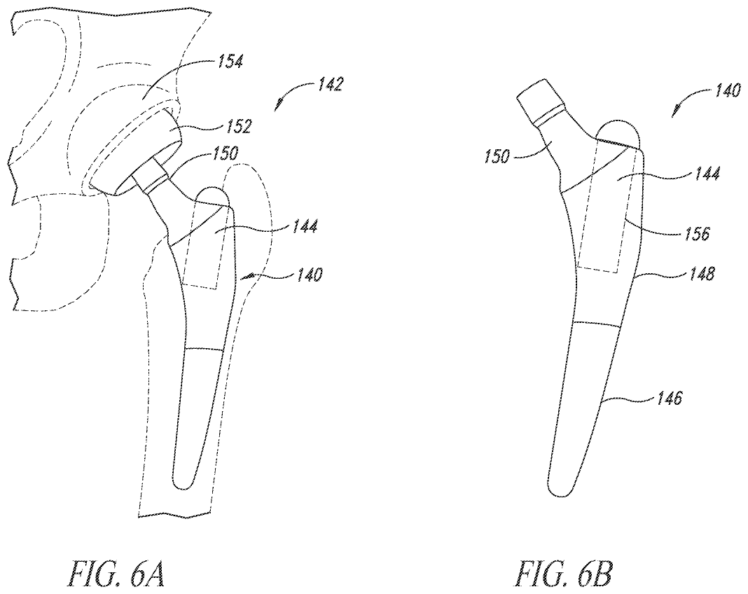

With reference to FIGS. 6A and 6B, an intelligent implant 140 may be part of a hip implant system 142. In this embodiment, the intelligent implant 140 corresponds to a femoral component of a hip replacement system for a THA and includes an implantable reporting processor (IRP) 144. The femoral component 140 includes a femoral stem 146, a femoral body 148, and a femoral neck 150. The femoral neck 150 is configured to physically attach to a femoral head 152 that is configured to attached to an acetabular cap 154. The femoral body 148 includes a receptacle 156 configured to receive the implantable reporting processor 144.

### Implantable Reporting Processors

The present disclosure provides implantable reporting processors (IRP) for implant systems that replace a joint. Embodiments disclosed include IRPs for knee implant systems, IRPs for shoulder implant systems, and IRPs for hip implant systems. As previously mentioned, in some embodiments the IRP is a component assembly that is manufactured independent of other components of the implant system and later assembled together with a component of an implant system. In some embodiments, the IRP is integrated with a component of the implant system during manufacture of the component.

### Component-Assembly Implantable Reporting Processors

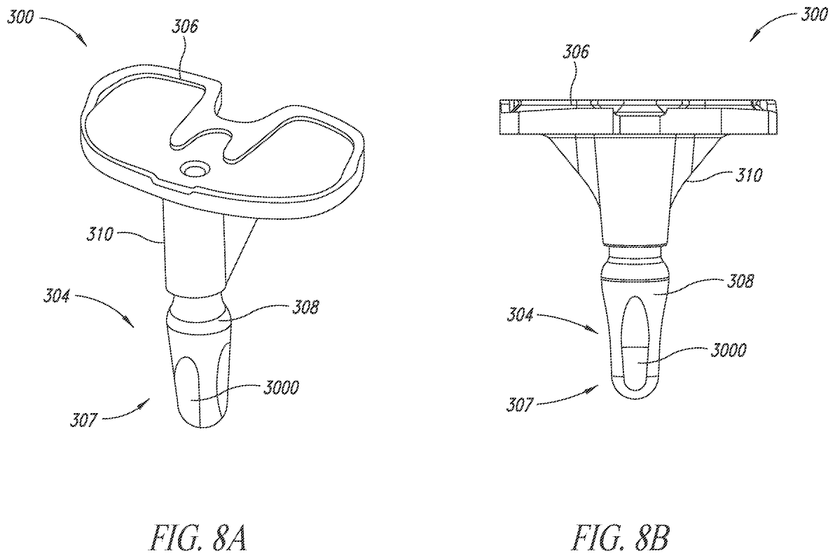

FIG. 8A is a perspective view of an embodiment of an intelligent implant in the form of a tibial component 300 of a knee prosthesis. FIGS. 8B and 8C are respectively, a front view and a side view of the tibial component 300 of FIG. 8A. The tibial component 300 can be the same as or similar to the embodiments of the tibial component 300 illustrated in and described in relation to FIGS. 3A and 3B. The tibial component 300 includes a tibial plate 306 with a tibial stem 310 and an implantable reporting processor 304 coupled to the tibial stem 310 to function as a tibial stem extension 308.

The implantable reporting processor 304 includes a distal portion 307 and a proximal portion (not visible). The proximal portion is positioned within the tibial stem 310 while the distal portion 307 extends from the tibial stem. As such, the distal portion 307 is exposed to the anatomy when the tibial component 300 is implanted. In the illustrated configuration, the distal portion 307 includes flutes 3000 that can enhance fixation of the tibial stem extension 308 with the bone material of the tibia. To this end, the flutes 3000 are recesses configured to receive bone cement to provide better fixation of the tibial component. In some configurations, as shown in FIG. 8C, the distal portion 307 of the implantable reporting processor 304 can have a length L₄ of between about 10 mm and about 60 mm, between about 20 mm and about 50 mm, between about 30 mm and about 40 mm, or about 30 mm.

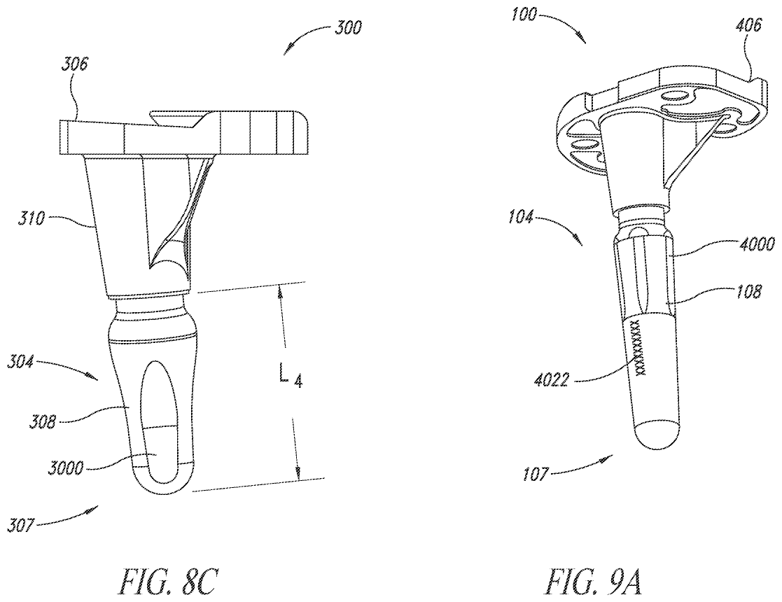

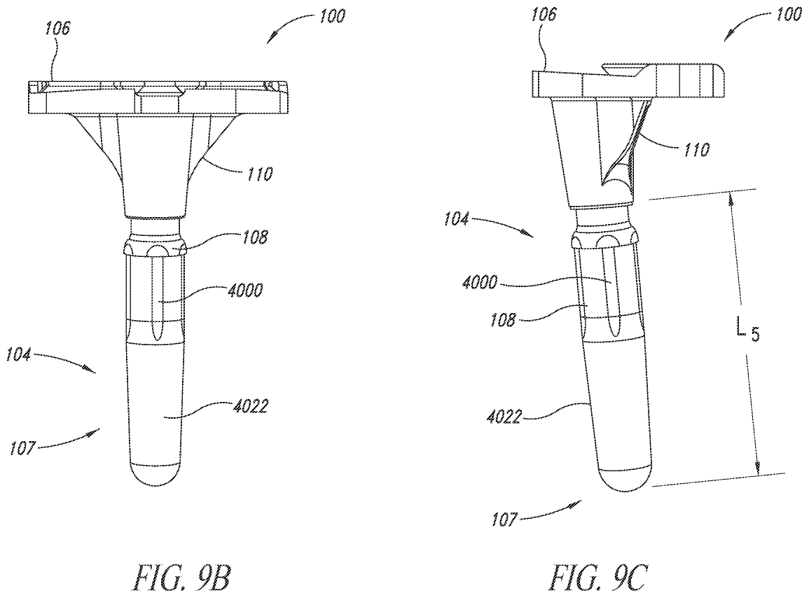

FIG. 9A is a perspective view of an embodiment of an intelligent implant in the form of a tibial component 100 of a knee prosthesis. FIGS. 9B and 9C are respectively, a front view and a side view of the tibial component 100 of FIG. 9A. The tibial component 100 can be the same as or similar to the embodiments of the tibial component 100 illustrated in and described in relation to FIGS. 2A and 2B. The tibial component 100 includes a tibial plate 106 and an implantable reporting processor 104 coupled to a tibial stem 110 to function as a tibial stem extension 108.

The implantable reporting processor 104 includes a distal portion 107 and a proximal portion (not visible). The proximal portion is positioned within the tibial stem 110 while the distal portion 107 extends from the tibial stem. As such, the distal portion 307 is exposed to the anatomy when the tibial component 300 is implanted. In the illustrated configuration, the distal portion 107 includes ribbings 4000 that can enhance the engagement of the tibial stem extension 108 with the bone material of the tibia. In some configurations, distal portion 107 can include a cover 4022. To this end, the ribbings 4000 may be spines configured to cut/engage with the bone material and allow for cementless use of the tibial component 300. In some configurations, as shown in FIG. 9C, the distal portion 107 can have a length L₅ of between about 30 mm and about 90 mm, between about 40 mm and about 80 mm, between about 50 mm and about 70 mm, or about 58 mm.

As previously described, the embodiments of the tibial component 100, 300 can include one or more parts. For example, the tibial components 100, 300 can include the tibial plate 106, 306, and the implantable reporting processor 104, 304. In some configurations the tibial plate 106, 306 can be integral with or separate from the implantable reporting processor 104, 304. In the configurations with the tibial plate 106, 306 being separate from the implantable reporting processor 104, 304, the tibial component 100, 300 can be coupled via a press-fit engagement, threaded engagement, snap-fit engagement, or other mechanical engagement.

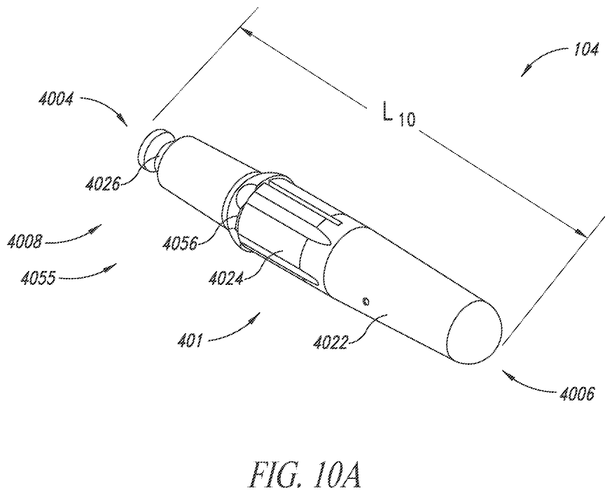

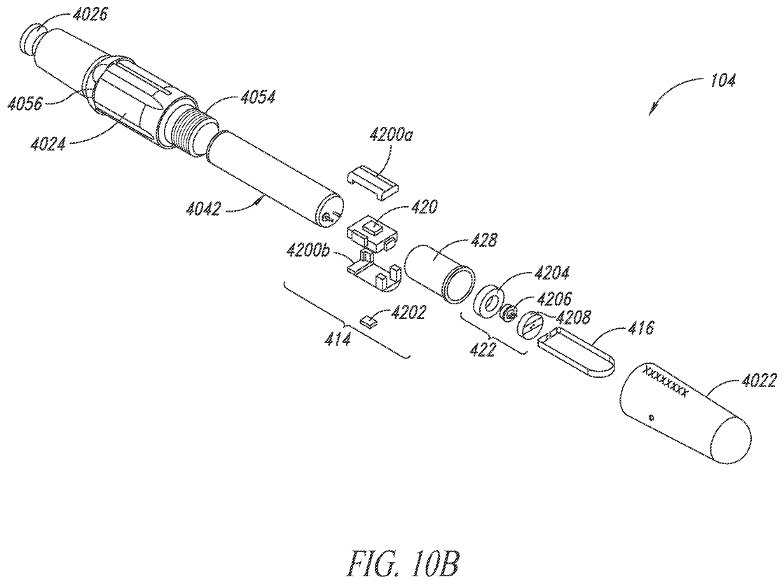

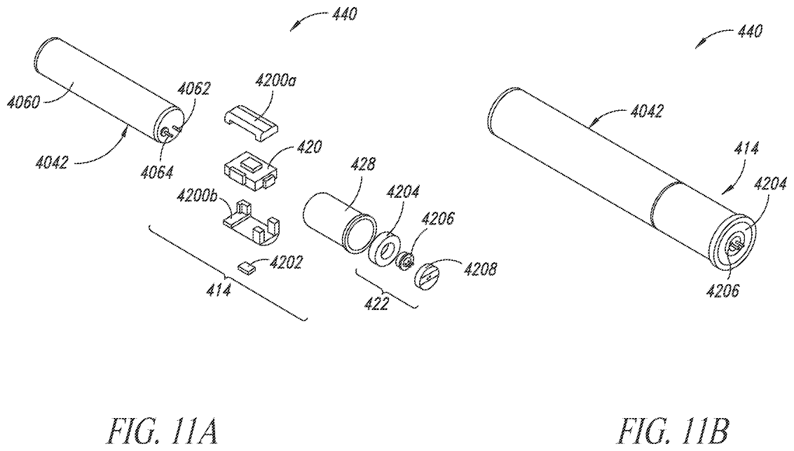

FIG. 10A illustrates a perspective view of the implantable reporting processor 104 and FIG. 10B illustrates an exploded view of the implantable reporting processor 104. The implantable reporting processor 104 includes a housing 401. The housing 401 can include a total length L₁₀ of between about 40 mm and about 110 mm, about 50 mm and about 100 mm, about 60 mm and about 90 mm, about 70 mm and about 80 mm, or about 78 mm. The housing 401 can include the cover 4022 and a casing 4055. In some configurations, the cover 4022 can be made from any material or combination of materials, such as plastic or ceramic, which allows radio-frequency (RF) signals to propagate through the cover 4022 with acceptable levels of attenuation and other signal degradation.

In some configurations, the casing 4055 can include a proximal portion 4008 including the proximal end 4004 and a coupling section 4026, and a body portion 4024 including a shoulder 4056. As shown in the illustrated configuration, the body portion 4024 of the casing 4055 can be positioned between the proximal portion 4008 and the cover 4022 when the cover 4022 is coupled to the casing 4055. In some configurations, the shoulder 4056 can include a distal facing surface configured to engage with the proximal end 14, 24 of the impaction sleeve 10, 20. The casing 4055 can be made from any material or combination of materials, such as a metal (e.g., titanium). The shoulder 4056 can comprise a maximum diameter of the implantable reporting processor 104.

As described below in the Impaction Sleeve section of the disclosure, the shoulder 4056 can be configured to engage with an impaction sleeve. When coupling the implantable reporting processor 104 to the tibial plate 106, a user can insert the coupling section 4026 of the implantable reporting processor 104 into the tibial plate 106. The user can secure the implantable reporting processor 104 to the tibial plate 106 using the impaction sleeve. For example, the user can position the impaction sleeve over the implantable reporting processor 104 (e.g., the distal portion of the implantable reporting processor 104 that includes at least the cover 4022) until a proximal end of the impaction sleeve abuts the shoulder 4056. The user can apply impaction forces to a distal head of the impaction sleeve until the implantable reporting processor 104 is securely coupled to the tibial plate 106.

As shown in FIG. 10B, the housing 401 encloses a battery 4042, an electronics assembly 414, a header assembly 422, and an antenna 416. The electronics assembly 414 can includes a circuit assembly 420, at least one liner 4200a, 4200b, an identification tag 4202, and a sleeve 428. The header assembly 422 can include a flange 4204, a feedthrough 4206, and a spacer 4208. The battery 4042 can be any suitable battery, such as a Lithium Carbon Monofluoride (LiCFx) battery, or other storage cell configured to store energy for powering the electronics assembly 414 for an expected lifetime (e.g., 5-25+ years) of the intelligent implant.

FIGS. 11A and 11B respectively illustrates a perspective exploded view and a perspective assembled view of a subassembly 440 of the implantable reporting processor 104 of FIGS. 10A and 10B. The subassembly 440 can include the battery 4042, the electronics assembly 414, and the header assembly 422. The battery 4042 has a lithium-carbon-monofluoride (LiCFx) chemistry, a cylindrical housing, hereinafter a cylindrical container, 4060, a cathode terminal 4062, and an anode terminal 4064, which is a plate that surrounds the cathode terminal. LiCFx is a non-rechargeable (primary) chemistry, which is advantageous for maximizing the battery-energy storage capacity. The cathode terminal 4062 makes conductive contact with an internal cathode electrode and couples to the cylindrical container using a hermetic feed-through insulating material of glass or ceramic. The use of the hermetic feed through prevents leakage of internal battery materials or reactive products to the exterior battery surface. Furthermore, the glass or ceramic feed-through material electrically insulates the cathode terminal 4062 from the cylindrical container 4060, which makes conductive contact with the internal anode electrode. The anode terminal is welded to the cylindrical container 4060. By locating the cathode and anode terminals 4062 and 4064 on the same end of the battery 4042, both terminals can be coupled to the electronics assembly 414 without having to run a lead, or other conductor, to the opposite end of the battery.

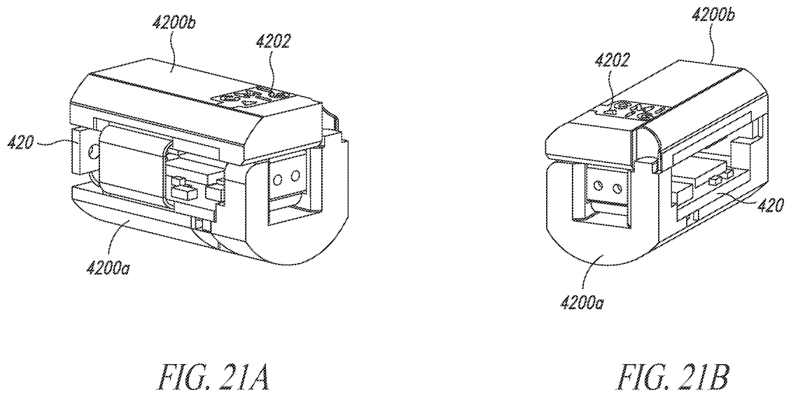

Regarding the electronics assembly 414, the liner 4200a, 4200b can be configured to enclose the circuit assembly 420. For example, the liner 4200a, 4200b can fully or at least partially enclose the circuit assembly 420. In some configurations, the at least one liner 4200a, 4200b is monolithic with two sections 4200a, 4200b. The illustrated configuration has a first liner 4200a and a second liner 4200b which engage with opposite sides of the circuit assembly 420. The first liner 4200a or the second liner 4200b can receive the identification tag 4202 on an outer surface of the liner. The identification tag 4202 may be radiopaque and can contain identifying information about the tibial component 400 and/or the patient such that a user can scan the tag from outside the patient's body by x-ray and the identifying information can be received by an external computing system. In some configurations, the identification tag 4202 can be radiopaque. The liners 4200a, 4200b and the circuit assembly 420 are further described below in relation to FIGS. 21A-23D.

The circuit assembly 420 and the liners 4200a, 4200b are configured to be enclosed by the sleeve 428. The outer surface(s) of the at least one liner 4200a, 4200b is configured to abut the inner surface of the sleeve 428 such that the circuit assembly 420 is secured within the sleeve 428. To this end, the liners 4200a, 4200b may have one or more rounded surfaces corresponding to the sleeve 428. The sleeve 428 may hold the one or more liners 4200a, 4200b together. In other configurations, the liners 4200a, 4200b may be directly attached to each other or indirectly attached to each other, for example by the circuit assembly 420, and reinforced by the sleeve 428. The sleeve 428 can include a distal opening configured to receive the flange 4204 and the feedthrough 4206 of the header assembly 422. In some configurations, the flange 4204 includes an opening configured to receive the feedthrough 4206. The feedthrough 4206 can be comprised of insulating material(s), such as glass and/or ceramic. The feedthrough 4206 can allow the electronics assembly 414 to communicate with the antenna 416 (not shown). In some configurations, the flange 4204 may be made from a metal or a combination of metals, such as titanium.

The sleeve 428 may be hermetically sealed at both ends to provide a hermetically sealed chamber that encloses the circuit assembly 420. For example, at the end of the sleeve 428 coupled to the header assembly 422, the flange 4204 may be welded to the sleeve and the feedthrough 4206 may be welded to the flange 4204 to create a hermetic seal. At the end of the sleeve 428 coupled to the battery 4042, the sleeve may be welded to the battery. In one embodiment, the atmosphere that is present in the hermetically sealed chamber is an inert atmosphere. In one embodiment, the atmosphere that is present in the chamber created by the hermetic seal has little or no moisture, i.e., little or no water vapor. For example, in embodiments, the water vapor in the atmosphere within the hermetically sealed chamber is less than 1%, or less than 0.5%, or less than 0.1%, or less than 0.01%, or less than 50 ppm, or less than 20 ppm, or less than 10 ppm water in the atmosphere. In one embodiment, the atmosphere present in the hermetically sealed chamber created by the hermetic seal has little or no oxygen. For example, in embodiments, the oxygen in the atmosphere within the hermetically sealed chamber is less than 15%, or less than 10%, or less than 5%, or less than 4%, or less than 3%, or less than 2%, or less than 1%, or less than 0.1%, or less than 0.01%, or less than 1,000 ppm, or less than 500 ppm, or less than 200 ppm, or less than 100 ppm, or less than 10 ppm oxygen in the atmosphere.

In embodiments, the atmosphere within the hermetically sealed chamber that encloses the circuit assembly 420 is primarily an inert gas selected from helium, neon, argon, krypton, and xenon, including mixtures thereof. For example, in one embodiment the atmosphere within the hermetically sealed chamber is primarily helium. For example, in one embodiment the atmosphere within the hermetically sealed chamber is primarily neon. For example, in one embodiment the atmosphere within the hermetically sealed chamber is primarily argon. For example, in one embodiment the atmosphere within the hermetically sealed chamber is primarily krypton. For example, in one embodiment the atmosphere within the hermetically sealed chamber is primarily xenon. In embodiments, when the atmosphere present in the hermetically sealed chamber is primarily an inert gas, then the identified inert gas or gases constitutes at least 50 molar percent (mol %) of the gas molecules in the enclosed atmosphere, or at least 60 mol %, or at least 70 mol %, or at least 80%, or at least 90 mol %, or at least 95 mol %, or at least 96 mol %, or at least 97 mol %, or at least 98 mol %, or at least 99 mol % of the gas molecules present in the enclosed atmosphere.

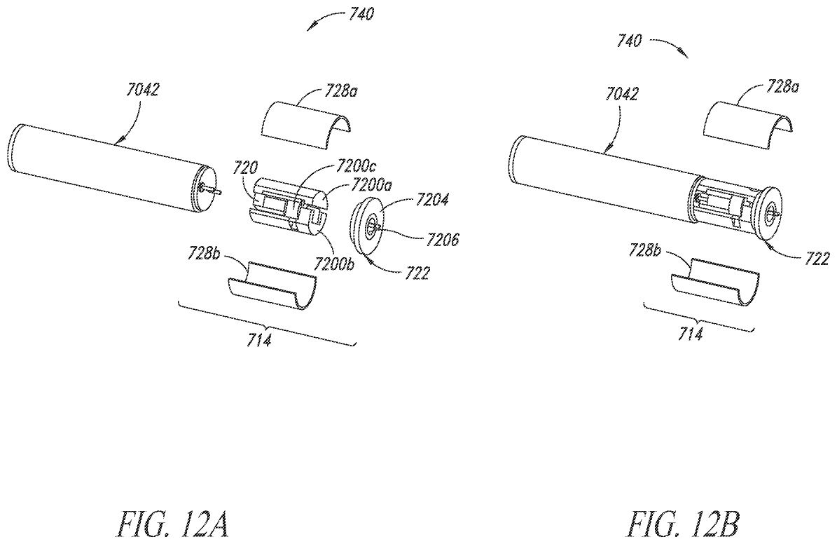

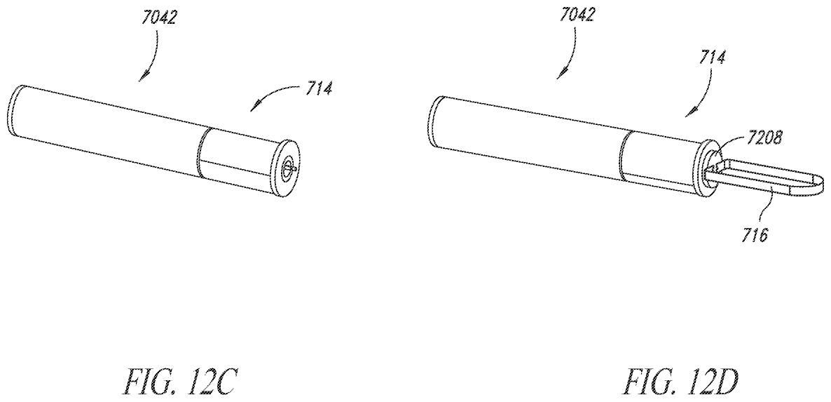

FIGS. 12A and 12B respectively illustrate a perspective exploded view and a partially exploded view of another embodiment of a subassembly 740 of the implantable reporting processor 104 of FIGS. 10A and 10B. FIG. 12C illustrates a perspective assembled view of the subassembly 740. FIG. 12D illustrates a perspective view of the subassembly 740 with an antenna 716. The subassembly 740 can include a battery 7042, an electronics assembly 714, and a header assembly 722. The battery 7042 can be any suitable battery, such as a Lithium Carbon Monofluoride (LiCFx) battery, or other storage cell configured to store energy for powering the electronics assembly 714 for an expected lifetime (e.g., 5-25+ years) of the intelligent implant. The battery 7042 may be configured the same as the battery 4042 described above with reference to FIG. 11A.

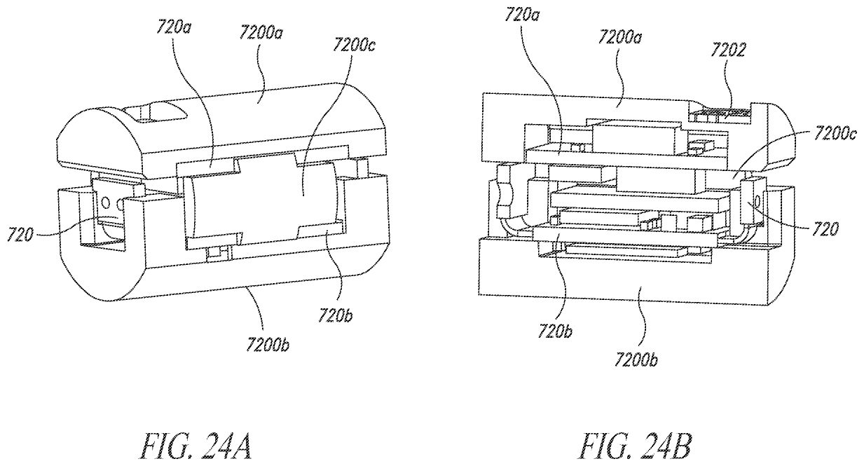

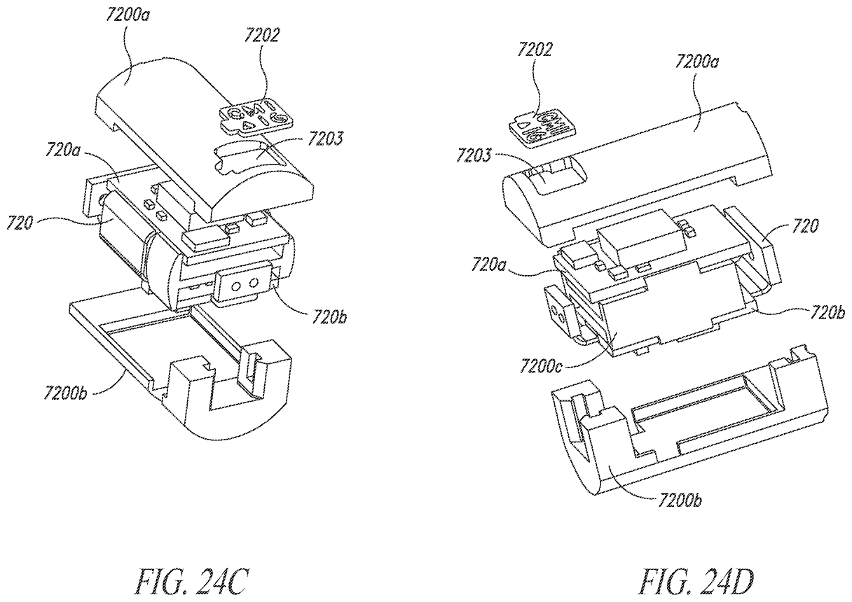

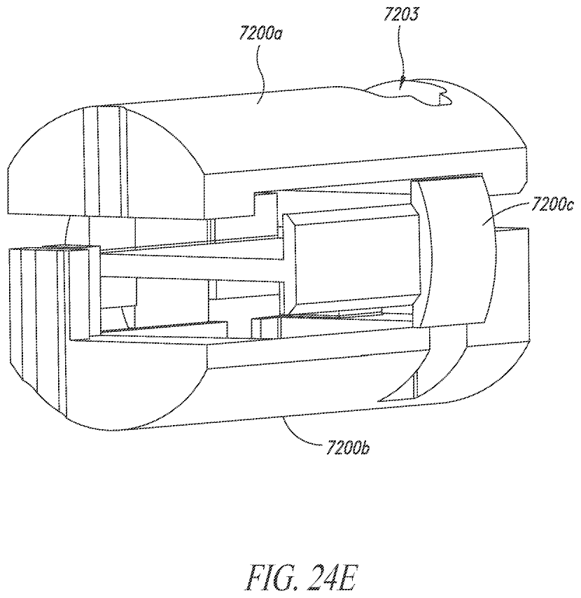

Regarding the electronics assembly 714, the sleeve may include a first portion 728a and a second portion 728b. The first portion 728a and the second portion 728b can be combined to enclose the at least one liner 7200a, 7200b, 7200c and the circuit assembly 720. In some configurations, the at least one liner 7200a, 7200b, 7200c can include a first liner 7200a, a second liner 7200b, and a third liner 7200c. The first liners 7200a and the second liner 7200b can be configured to enclose the circuit assembly 720. The third liner 7200c can be positioned in the middle of the circuit assembly 720. The at least one liner 7200a, 7200b, 7200c and the circuit assembly 720 are further described below in relation to FIGS. 24A-24E.

In one embodiment, the present disclosure provides an implantable reporting processor that includes a casing and an electronics assembly at least partially enclosed by the casing. The electronics assembly includes a circuit assembly coupled to a battery that provides power to the implantable reporting processor. The circuit assembly includes circuitry, e.g., one or more sensors, configured to generate data related to the implantable reporting processor. The implantable reporting processor further includes a liner. The liner may comprise a first section configured to receive a first portion of the circuit assembly and a second section configured to receive a second portion of the circuit assembly. In some embodiments, the circuit assembly is flexible and foldable. When the circuit assembly is flexible circuitry and is folded, the liner partially encloses the circuit assembly. The implantable reporting processor may include a sleeve configured to enclose the circuit assembly. The sleeve comprises a distal rim configured to abut a proximally facing surface of the casing. The implantable reporting processor may include an antenna coupled to the circuitry and configured to transmit the data from the circuitry. With reference to FIG. 10B, in one embodiment the sleeve 428 is a unitary (monolithic) structure. With reference to FIGS. 12A and 12B, in another embodiment the sleeve is formed from two sections 728a, 728b. When the sleeve is formed from two or more sections, those multiple sections may be welded together. When the sleeve is a unitary structure, it does not contain any welding to hold multiple sections together.

In one embodiment, the present disclosure provides an implantable reporting processor that includes a casing and an electronics assembly at least partially enclosed by the casing. The electronics assembly includes a circuit assembly coupled to a battery that provides power to the implantable reporting processor. The circuit assembly includes circuitry, e.g., one or more sensors, configured to generate data related to the implantable reporting processor. The implantable reporting processor further includes a sleeve 428 configured to enclose the circuit assembly. The sleeve 428 includes a distal rim 429 configured to abut a proximally facing surface of the casing. With reference to FIG. 10B, in one embodiment the sleeve 428 is a unitary (monolithic) structure. With reference to FIGS. 12A and 12B, in another embodiment the sleeve is formed from two sections 728a, 728b. When the sleeve is formed from two or more sections, those multiple sections may be welded together. When the sleeve is a unitary structure it does not contain any welding to hold multiple sections together.

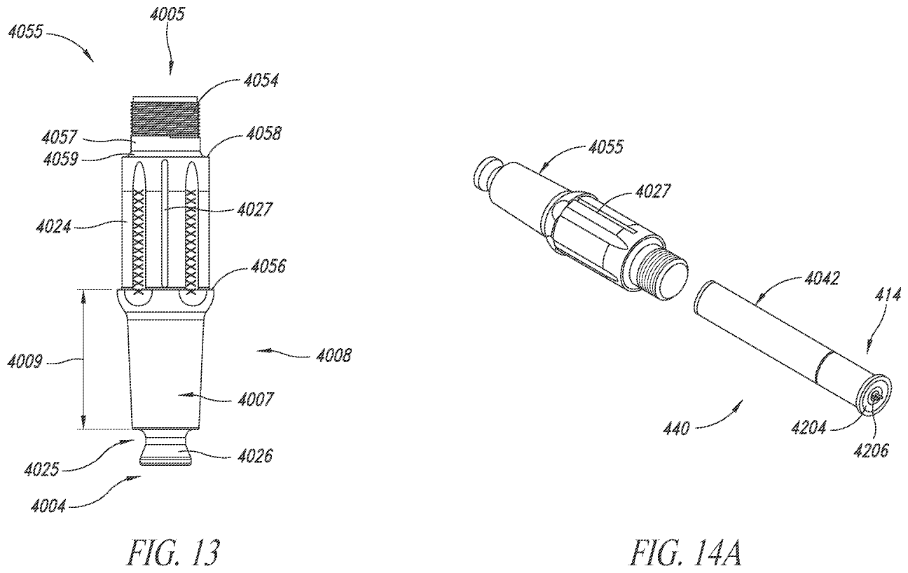

FIG. 13 is a side view of the casing 4055 of the implantable reporting processor 104 of FIG. 10A. As described above, the casing 4055 can include a proximal portion 4008 including a proximal end 4004 and a coupling section 4026, and a body portion 4024 including a shoulder 4056.

In one embodiment, which is illustrated in FIG. 13, the proximal portion 4008 of the casing 4055 is configured to fit within a receptacle 112 of a tibial plate 106 of a knee prosthesis, such as shown in FIG. 2B, and serves to secure the implantable reporting processor 104 to the tibial plate. As shown in FIG. 13, the coupling section 4026 may include an indent 4025 that is annularly symmetrical around the perimeter of the coupling section. This indent 4025 is configured to accept a set screw which would extend through a hole in the tibial plate to thereby aid in securing the casing 4055 of the implantable reporting processor 104 to the tibial plate. Because the indent is annularly symmetrical, the casing 4055 need not be oriented in any particular direction relative to the tibial plate in order for the set screw to extend through a side of the tibial plate and into the indent 4025. In addition, the proximal portion 4008 may include a smooth tapered surface 4007 that is symmetrical around the perimeter of the proximal portion 4008 and extends for a distance 4009 between the shoulder 4056 and the coupling section 4026. This surface 4007 provides a trunnion for a machine taper connection to the receptable of the tibial plate. In one embodiment, the proximal portion 4008 of the casing 4055 includes both of an annularly symmetric indent 4025 and a smooth tapered surface 4007, where each of these features independently aid in securing the implantable reporting processor 104 to a tibial plate.

Thus, in one embodiment the present disclosure provides an implantable reporting processor that includes a casing having a shoulder and a proximal end and a proximal portion located between the shoulder and the proximal end of the casing. The proximal portion includes a coupling region with an indent that is annularly symmetrical around a perimeter of the coupling section, and a smooth tapered surface that is symmetrical around the perimeter of the proximal portion. The smooth tapered surface provides a trunnion for a machine taper connection to the tibial plate and the annularly symmetrical indent provides a recess for a set screw from the tibial plate.

Still referring to FIG. 13, in some configurations, the body portion 4024 of the casing 4055 can include one or more alignment features 4027. For example, the illustrated configuration shows an alignment feature 4207 that extends longitudinally along the body portion 4024. The alignment feature 4207 can be a line that was etched or otherwise formed onto an outer surface of the body portion 4024 by a laser or other etching tool. The alignment feature 4207 can be configured to engage with one or more corresponding alignment features of one or more of the other components of the tibial component 400.

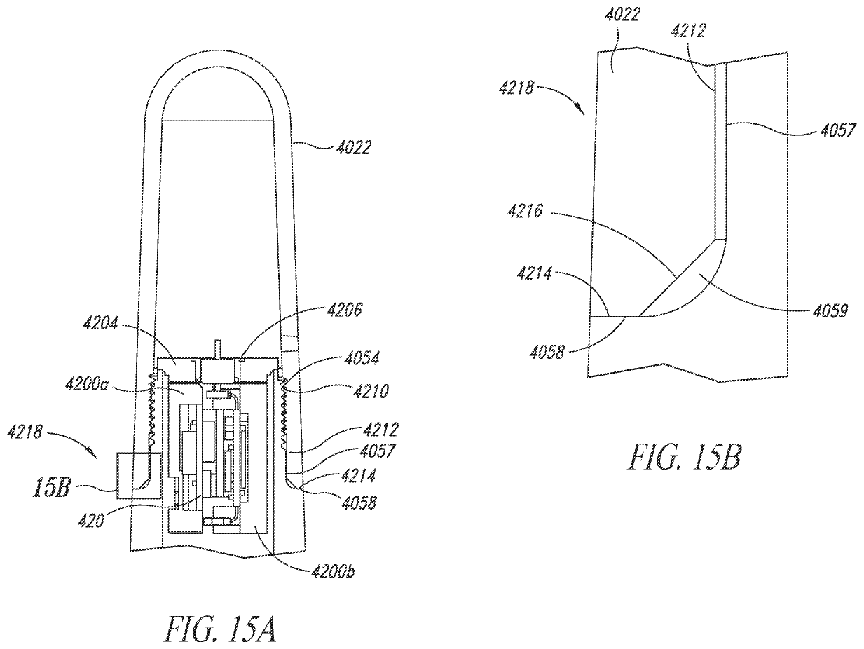

The casing 4055 can also include a distal end 4005 opposite the proximal end 4004, a threaded surface 4054 near or adjacent the distal end 4005, a smooth surface 4057, and a distal facing wall 4058. In some configurations, the casing 4055 can include a tapered surface 4059 between the smooth surface 4057 and the distal facing wall 4058. As further described below in relation to FIGS. 15A-15B, the threaded surface 4054 and the distal facing wall 4058 can be configured to engage with the cover 4022.

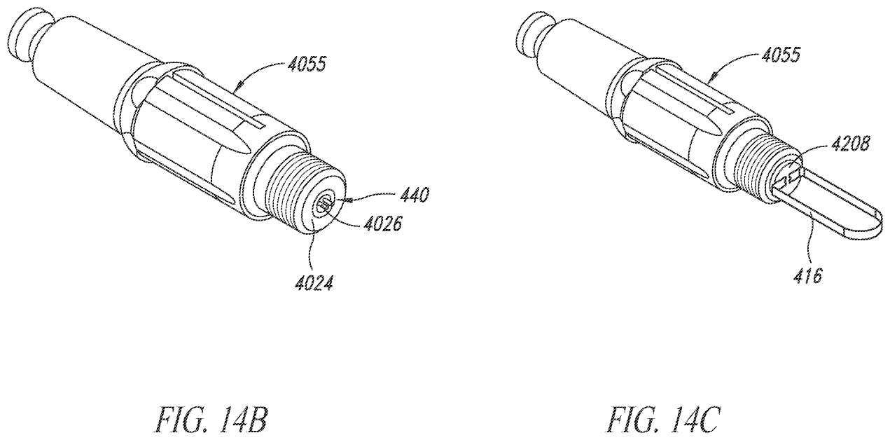

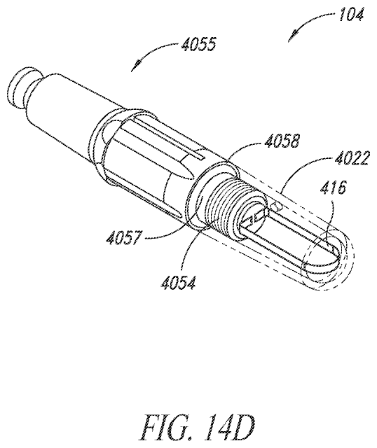

FIG. 14A is a perspective view of the casing 4055 and a subassembly. The subassembly may be the subassembly 440 of FIG. 11B or the subassembly 740 of FIG. 12C. In either embodiment, the casing 4055 is configured to receive the subassembly 440, 740.

Considering for example the subassembly 440 of FIG. 11B, in some configurations the battery 4042 of the subassembly includes at least one alignment feature (not shown) configured to correspond with the alignment feature 4207 of the casing 4055. For example, during assembly, the alignment feature of the battery 4042 can be aligned with the alignment feature 4027 of the casing 4055 so that the electronics assembly 414 is properly aligned within the casing 4055.

FIG. 14B is a perspective view of the subassembly 440 enclosed by the casing 4055. FIG. 14C is a perspective view of the assembly shown in FIG. 14B with a spacer 4208 and an antenna 416 attached. FIG. 14D is a perspective view of the casing 4055 with subassembly 440 and antenna 416 shown in FIG. 14C with a cover 4022 attached to the casing to enclose the antenna with the cover 4022 transparent so that the internal components can be seen.

FIGS. 15A and 15B illustrates partially cross-sectional views of a cover 4022 coupled to a casing 4055. As shown in FIG. 15A, the cover 4022 can include a closed end and an open end configured to receive a portion of the casing 4055. The cover 4022 can include a proximal fillet 4218 adjacent the open end. The proximal fillet 4218 can include a proximal edge 4214. The cover 4022 can include a threaded portion 4210 and a smooth portion 4212 positioned between the threaded portion 4210 and the proximal edge 4214 such that the threaded portion 4210 is spaced apart from the proximal edge 4214. The proximal fillet 4218 can include the smooth portion 4212. The threaded portion 4210 of the cover 4022 can be configured to engage with the threaded surface 4054 of the casing 4055. In some configurations, the proximal facing surface of the proximal edge 4214 can be configured to abut the distal facing wall 4058 of the casing 4055 when the cover 4022 is coupled to the casing 4055.

FIG. 15B illustrates an enlarged cross-sectional view of the proximal fillet 4218 of the cover 4022. For example, the proximal fillet 4218 can include a tapered portion 4216 positioned between the smooth portion 4212 and the proximal edge 4214. In some configurations, the proximal fillet 4218 is configured so that there are only two engagement points between the cover 4022 and the casing 4055. For example, the two engagement points can include the engagement between the threaded portion 4210 of the cover 4022 and the threaded surface 4054 of the casing 4055, and the engagement between the proximal edge 4214 of the cover 4022 and the distal facing wall 4058 of the casing 4055. Advantageously, this arrangement strengthens the tibial component 400 and reduces or eliminates any sharp edges of the tibial component 400. Additionally, this configuration can improve the ability of the cover 4022 to withstand forces from contacting the bone of the patient. In some configurations, the cover 4022 and the casing 4055 may have one engagement point or more than two engagement points. In some configurations, an O-ring is not present between the cover and the casing, i.e., the cover and the casing are configured such that there is no place to put an O-ring between the cover and the casing.

In one embodiment, the cover 4022 is sufficiently strong that it can withstand anatomical fatigue loading that result from forces exerted on the cover upon contacting the bone of the patient during patient activity. In other words, the cover can stand up to pressure being exerted against the exterior surface of the cover such that the cover does not break or deform in response to that pressure. In one embodiment, the cover is formed from a thermoplastic, where in one embodiment the thermoplastic is selected to have an impact strength such that repeated hitting of the cover at a torque 25-35 N-meters, e.g., 25, or 26, or 27, or 28, or 29, or 30, or 31, or 32, or 33, or 34, or 35 N-meters, does not break the cover, where the number of times the cover is impacted at this torque is at least one million times, e.g., two, or four, or six, or eight, or ten million times. The strikes against the cover are measured from the inferior surface of the tibial plate.

In one embodiment, the cover 4022 is formed from a thermoplastic, where exemplary thermoplastics include polyether ether ketone (PEEK), acrylonitrile butadiene styrene (ABS), and polysulfone. In one embodiment, the cover 4022 comprises a plastic having a Shore D hardness in a range of 20 to 100. In one embodiment, the cover 4022 comprises an outer surface that faces towards a tibia of subject in which the implantable medical device is implanted, and an inner surface that faces towards the cavity, where the distance between the outer surface and the inner surface is between 1.0 and 1.5 mm and the maximum diameter between opposing outer surfaces is between 12 and 16 mm, or is between 12 and 15 mm.

In one embodiment, the entire outer surface of the cover is smooth. In one embodiment, the entire inner surface is smooth with the exception of the threaded portion 4210 of the inner surface. Thus, in one embodiment, the cover does not include any ribbing or other sort of support structure. When the inner and outer surfaces are largely and optionally entirely (with the exception of the threaded portion 4210) smooth, then the cover avoids the presence of a stress concentrator that may reduce the strength of the cover to repeated forces such as occurs when the cover is located in a subject's tibia.

For example, in one embodiment the present disclosure provides a cover 4022 comprising PEEK, optionally made entirely from PEEK, where the outer surface of the cover is smooth and the inner surface of the cover is smooth except for the presence of the threaded portion 4210, where the thickness of the cover, i.e., the distance between the inner and outer surfaces, is in the range of 1.0 to 1.5 mm, and the length of the cover, i.e., the distance from the proximal end to the distal end of the cover, is in the range of 30-40 mm, and the width of the cover, i.e., the further distance between opposing outer surfaces, is in the range of 12-15 mm. The impact strength of the cover may be increased by increasing the thickness of the cover however this may result in either a cover that is undesirably wide for placement in a subject's tibia, and/or in a cover cavity that is undesirably narrow for placement of an antenna of the present disclosure. As shown in, e.g., FIG. 15A, the cover may have a rounded distal end (nosecone) and an open proximal end (base). In cross-section across the longitudinal axis of the cover, the inner and outer surfaces may be circular. A cover of the present disclosure may have a fatigue strength such that cyclic loading of the cover at a torque 25-35 N-meters does not break the cover, where the number of times the cover is loaded at this torque is up to about 10 million times, where the applied torque against the cover is measured from the inferior surface of the tibial plate.

In one embodiment the present disclosure provides an implantable reporting processor that includes an electronics assembly, an antenna, a casing configured to house at least a portion of the electronics assembly, and a cover configured to house the antenna. The casing and the cover abut one another and form an enclosed space that houses the antenna and a filler. In one embodiment, the present disclosure provides that the enclosed space of the cover contains both an antenna and a filler. In one embodiment, the filler is a solid material, i.e., a material that does not flow at room temperature. In one embodiment, the solid filler is an organic polymer such as a thermoset resin. In one embodiment the organic polymer is an epoxy resin or a silicone resin. In one embodiment the filler is recognized as medical grade. In one embodiment, the solid material has a high hardness as measured on the Shore D scale, for example a hardness of greater than 50, or greater than 60, or greater than 70, or greater than 75, or greater than 80 Shore D hardness. In one embodiment, the solid material has high electrical insulating properties, for example a dielectric constant of from 1.5 to 10, or from 2.5 to 6. In one embodiment, the solid material has a low elongation as measured as %, such as below 20%, or below 15%, or below 10%. In one embodiment, the solid material has a high tensile strength, as measured in psi, such as at least 8,000, or at least 7,500, or at least 7,000, or at least 6,500, or at least 6,000. In one embodiment, the solid material has a high modulus, as measured in psi, such as a modulus of greater than 300,000, or greater than 250,000, or greater than 200,000.

In one embodiment, the solid filler is an epoxy resin. The solid epoxy may be formed from a liquid epoxy resin that has been combined with a hardener, i.e., the liquid comprises a resin and a hardener which through curing react to provide the solid epoxy material. In one embodiment, the curing occurs at a temperature of about 25° C., or within a temperature of 20-30° C., which may be referred to as room temperature curing. In one embodiment, the curing condition for the liquid epoxy is a heat cure, where a heat cure includes curing that occurs at above room temperature, i.e., above about 30° C. In one embodiment, the solid epoxy is prepared from a liquid epoxy that has a low viscosity as measured at room temperature, e.g., a viscosity of less than 50,000 cps, or less than 40,000 cps, or less than 30,000 cps, or less than 20,000 cps, or less than 15,000 cps, or less than 10,000 cps. Thus, in one embodiment the solid epoxy resin is the reaction product of a liquid precursor comprising a liquid epoxy and a hardener, where the liquid precursor has a low viscosity as measured at room temperature so that it may be injected through a fill port of the cover and fill the enclosed space of the cover, e.g., a viscosity of less than 50,000 cps, or less than 40,000 cps, or less than 30,000 cps, or less than 20,000 cps, or less than 15,000 cps, or less than 10,000 cps, where the liquid precursor is thermally cured at room temperature within the cover. The cover may also have a bleed-valve port in addition to the fill port, where these two ports may be sealed using, e.g., ultrasonic welding, after the liquid precursor has filed the enclosed space of the cover.

The resulting solid epoxy may be characterized by one or more of hardness (Shore D), insulating properties (dielectric constant), elongation, tensile strength, modulus. In one embodiment, the solid epoxy has a high hardness as measured on the Shore D scale, for example a hardness of greater than 50, or greater than 60, or greater than 70, or greater than 75, or greater than 80 Shore D hardness. In one embodiment, the solid epoxy has good electrical insulating properties, for example a dielectric constant of 1.5 to 10, or of 2.5 to 6. In one embodiment, the solid epoxy has a low elongation as measured as %, such as below 20%, or below 15%, or below 10%. In one embodiment, the solid epoxy has a high tensile strength, as measured in psi, such as at least 8,000, or at least 7,500, or at least 7,000, or at least 6,500, or at least 6,000. In one embodiment, the solid epoxy has a high modulus, as measured in psi, such as a modulus of greater than 300,000, or greater than 250,000, or greater than 200,000. The cover that forms the cavity that is filled with epoxy resin may comprise PEEK.

In one embodiment, the solid material is a silicone resin. The solid silicone may be prepared from a two-component silicone that is a liquid and may be referred to as a liquid precursor. The liquid precursor may be injected into the enclosed space of the cover through a fill port of the cover, and then cured within the enclosed space to provide the solid silicone. The solid silicone may have a hardness as measured on the Shore D scale in the range of 25 to 50. The cover that forms the enclosed space that is filled with silicone resin may comprise PEEK.

As mentioned herein, in one embodiment the present disclosure provides an implantable reporting processor that includes an electronics assembly, an antenna, a casing configured to house at least a portion of the electronics assembly, and a cover configured to house the antenna. The casing and the cover abut one another and form an enclosed space that houses the antenna and a filler, where the enclosed space of the cover contains both an antenna and a filler. The electronics assembly may include a sensor that along with other electronic components is housed in a hermetically sealed chamber of the casing, where the hermetically sealed chamber may hold an atmosphere, i.e., a gas, in contact with the electronics components. Thus, the present disclosure provides an implantable reporting processor having two separate chambers, at least one of which is a hermetically sealed chamber. The implantable reporting processor includes an electronics assembly including a sensor, an antenna, a casing configured to house at least a portion of an electronics assembly within a hermetically sealed chamber. The implantable reporting processor also includes a cover configured to house the antenna. The casing and the cover abut one another and form a chamber (which may be referred to as the antenna chamber) that may or may not be hermetically sealed and that houses the antenna. Within embodiments, the atmosphere within the hermetically sealed chamber is primarily an inert gas selected from helium, neon, argon, krypton, and xenon, including mixtures thereof, which has a low moisture and low oxygen content as described herein, and the antenna chamber contains a filler as described herein. For example, in one embodiment the atmosphere within the hermetically sealed chamber is primarily helium. For example, in one embodiment the atmosphere within the hermetically sealed chamber is primarily neon. For example, in one embodiment the atmosphere within the hermetically sealed chamber is primarily argon. For example, in one embodiment the atmosphere within the hermetically sealed chamber is primarily krypton. For example, in one embodiment the atmosphere within the hermetically sealed chamber is primarily xenon. In embodiments, when the atmosphere present in the hermetically sealed chamber is primarily an inert gas, then the identified inert gas or gases constitutes at least 50 molar percent (mol %) of the gas molecules in the enclosed atmosphere, or at least 60 mol %, or at least 70 mol %, or at least 80%, or at least 90 mol %, or at least 95 mol %, or at least 96 mol %, or at least 97 mol %, or at least 98 mol %, or at least 99 mol % of the gas molecules present in the enclosed atmosphere.

### Integrated Implantable Reporting Processors

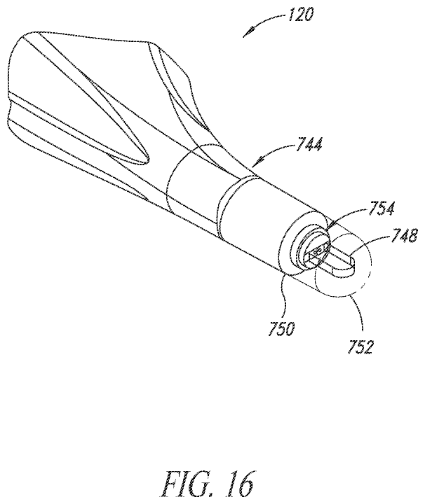

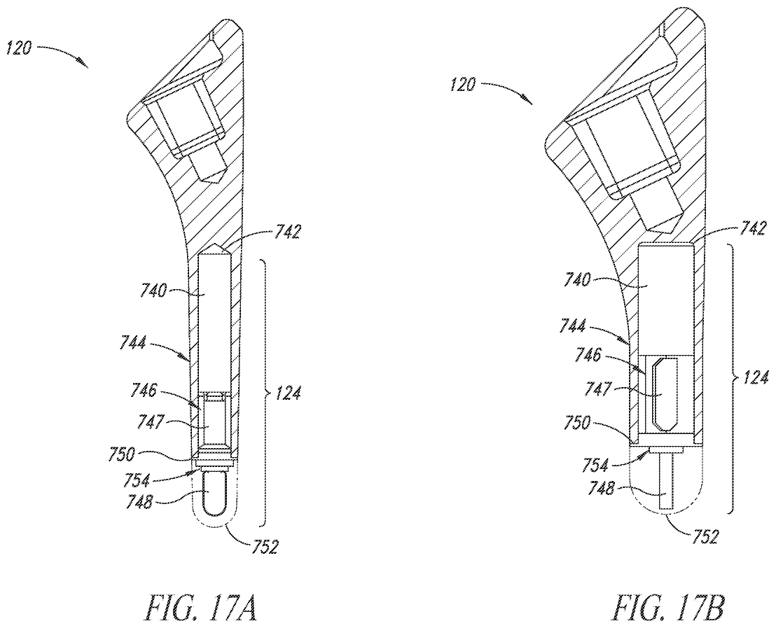

With reference to FIGS. 16, 17A and 17B, an implantable reporting processor 124 is integrated with a humeral component 120 of a shoulder implant system like that shown in FIG. 5A. The implantable reporting processor 124 can be integrated with humeral components of different sizes, including for example a standard size humeral component (shown in FIG. 17A) having a length in the range of 91 mm to 94 mm, and a micro size humeral component (shown in FIG. 17B) having a length in the range of 66 mm to 29 mm. In either configuration, the implantable reporting processor 124 includes a battery 740 configured to fit within a receptacle 742 formed in the humeral stem 744 portion of the humeral component 120, an electronics assembly 746 configured to fit within a receptacle, and an antenna 748 outside the receptacle that extends from the tip 750 of the humeral stem, and a cover 752 that covers the antenna. An antenna feedthrough 754 at the tip 750 of the humeral stem 744 extends partially into the receptacle 742.

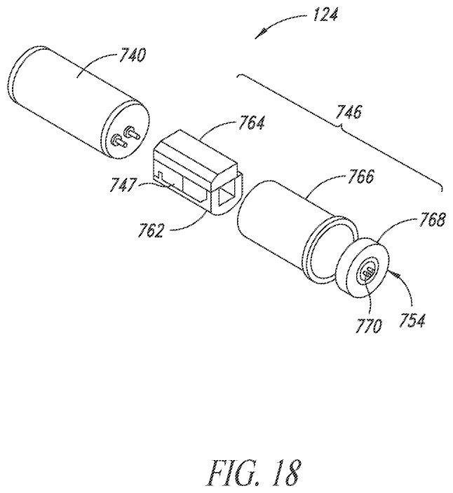

With reference to FIGS. 17A, 17B, and 18, the primary components of the implantable reporting processor 124 include the battery 740, the electronics assembly 746, and the antenna 748. The battery 740 can be any suitable battery, such as a Lithium Carbon Monofluoride (LiCFx) battery, or other storage cell configured to store energy for powering the electronics assembly 746 for an expected lifetime (e.g., 5-25+ years) of the intelligent implant.

The electronics assembly 746 includes a circuit assembly 747 that has one or more sensors and a processor configured to receive and process information from the sensors relating to the state and functioning of the implantable reporting processor 124 and the state of the patient within which the implantable reporting processor is implanted. The circuit assembly 747 of the electronics assembly 746 is further configured to transmit the processed information to an external device through the antenna 748. The circuit assembly 747 of the electronics assembly 746 may be configured as described below with reference to FIGS. 21A-23D.

With reference to FIG. 18, other components of the implantable reporting processor 124 include a first liner 762, a second liner 764, a sleeve 766, a flange 768, and an implant-grade bipolar feedthrough 770. The first liner 762, a second liner 764 function to mechanically stabilize the circuit assembly 747 within the sleeve 766. The first liner 762 and a second liner 764 can be formed from any suitable material, including for example, polycarbonate. The sleeve 766 can be formed of a biocompatible metallic material. In some embodiments, the material is titanium. The flange 768 is formed of a biocompatible metallic material. In some embodiments, the material is titanium. The antenna spacer 758 is formed of a non-conductive biocompatible material. In some embodiments, the antenna spacer 758 is formed of a PEEK. The feedthrough 770 is formed of a non-conductive biocompatible material. In some embodiments, the feedthrough 770 is formed of a ceramic.

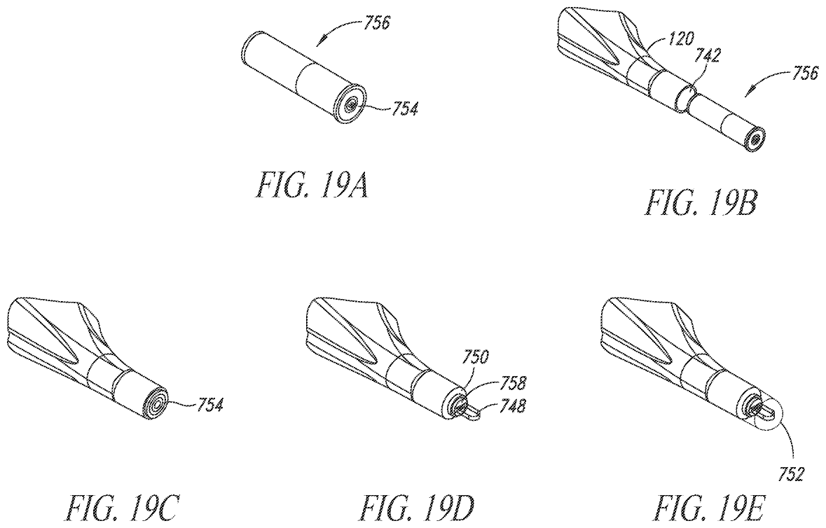

With reference to FIGS. 19A-19E, an intelligent implant for a shoulder implant system is assembled from a humeral component 120, a hermetic subassembly 756, an antenna 748, and a cover 752. With reference to FIGS. 19B and 19C, the hermetic subassembly 756 is placed in the receptacle 742 of the humeral component 120 and the distal end of the hermetic subassembly is hermetically welded to the distal end of the humeral component. With reference to FIG. 19D, an antenna spacer 758 is placed over the external pins of the feedthrough 754 (shown in FIG. 19C) of the hermetic subassembly 756, and an antenna 748 is welded to the feedthrough pins. The antenna spacer 758 is formed of a non-conductive biocompatible material. In some embodiments, the antenna spacer 758 is formed of a PEEK. With reference to FIG. 19E, a cover 752 is then assembled onto and secured to the tip 750 of the humeral component 120. In some embodiments, the cover 752 is back-filled with an epoxy (fill and bleed ports not shown). The epoxy material encapsulates the antenna 748 within the cover 752. The epoxy material may be medical grade silicone. Encapsulating the antenna 748 increases structural rigidity of the portion of implantable reporting processor 124 extending from the receptacle 742 of the humeral component 120 and isolates the antenna from tissue and body fluid.

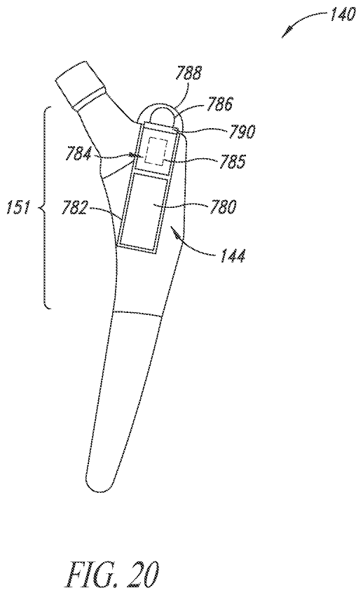

With reference to FIG. 20, an implantable reporting processor 144 is integrated with a femoral component 140 of a hip implant system like that shown in FIG. 6A. The implantable reporting processor 144 includes a battery 780 configured to fit within a receptacle 782 formed in the proximal, midline portion 151 of the femoral component 140, an electronics assembly 784 configured to fit within the receptacle, and an antenna 786 outside the receptacle that extends from a surface of the femoral component, and a cover 788 that covers the antenna. An antenna feedthrough 790 of the electronics assembly 784 is positioned at the surface of the femoral component 140 and extends partially into the receptacle 782. The battery 780 can be any suitable battery, such as a Lithium Carbon Monofluoride (LiCFx) battery, or other storage cell configured to store energy for powering the electronics assembly 784 for an expected lifetime (e.g., 5-25+ years) of the intelligent implant. The electronics assembly 784 is hermetically welded to the femoral component 140 thereby creating a hermetic chamber within the receptacle 782.

The electronics assembly 784 includes a circuit assembly 785 that has one or more sensors and a processor configured to receive and process information from the sensors relating to the state and functioning of the implantable reporting processor 144 and the state of the patient within which the implantable reporting processor is implanted. The electronics assembly 784 is further configured to transmit the processed information to an external device through the antenna 786. The circuit assembly 785 of the electronics assembly 784 may be configured as described below with reference to FIGS. 21A-23D.

In some embodiments, during assembly the cover 788 is back-filled with an epoxy (fill and bleed ports not shown). The epoxy material encapsulates the antenna 786 within the cover 788. The epoxy material may be medical grade silicone. Encapsulating the antenna 786 increases structural rigidity of the portion of implantable reporting processor 144 extending from the receptacle 782 of the femoral component 140 and isolates the antenna from tissue and body fluid.

With reference to FIGS. 4A and 4B, an implantable reporting processor 404 is integrated with a tibial component 400 of a knee implant system. The implantable reporting processor 404 includes a subassembly 409 configured to fit within a receptacle 412 formed in the tibial stem 410 portion of the tibial component. The subassembly 409 includes a battery 413, an electronics assembly 415, an antenna feedthrough 417, and an antenna 419. When the subassembly 409 is placed in the receptacle 412 the battery 413 and the electronics assembly 415 are located inside the receptacle 412, while the antenna 419 is positioned outside the receptacle. The antenna feedthrough 417 extends partially into the receptacle 412. The electronics assembly 415 portion of the subassembly 409 is hermetically welded to the tibial stem 410 thereby creating a hermetic chamber within the receptacle 412 within which the battery 413 and the electronics assembly 415 reside. A cover 411 covers the antenna 419 and may be secured to the tibial stem 410 through a threaded coupling. Alternatively, the cover 411 may be over molded to the subassembly 409 prior to placement of the subassembly in the receptacle 412. The antenna feedthrough 417 extends partially into the receptacle 412.

The battery 413 can be any suitable battery, such as a Lithium Carbon Monofluoride (LiCFx) battery, or other storage cell configured to store energy for powering the electronics assembly 415 for an expected lifetime (e.g., 5-25+ years) of the intelligent implant. The battery 413 may be configured the same as the battery 4042 described above with reference to FIG. 11A.

The electronics assembly 415 includes a circuity assembly that has one or more sensors and a processor configured to receive and process information from the sensors relating to the state and functioning of the implantable reporting processor 404 and the state of the patient within which the implantable reporting processor is implanted. The electronics assembly 415 is further configured to transmit the processed information to an external device through the antenna 419. The circuit assembly of the electronics assembly 415 may be configured as described below with reference to FIGS. 21A-23D.

In some embodiments, during assembly the cover 411 is back-filled with an epoxy (fill and bleed ports not shown). The epoxy material encapsulates the antenna 419 within the cover 411. The epoxy material may be medical grade silicone. Encapsulating the antenna 419 increases structural rigidity of the portion of implantable reporting processor 404 extending from the receptacle 412 of the tibial component 400 and isolates the antenna from tissue and body fluid.

Circuit Assembly

FIGS. 21A and 21B illustrate perspective views of the circuit assembly 420 enclosed by the at least one liner 4200a, 4200b. The at least one liner 4200a, 4200b can fully or at least partially enclose the circuit assembly 420. The at least one liner 4200a, 4200b are configured to mechanically stabilize the circuit assembly 420 and its associated sensors, e.g., accelerometers and gyroscopes. The at least one liner 4200a, 4200b are also configured to insulate the circuit assembly 420 from outside forces (e.g., shock or vibration). For example, the at least one liner 4200a, 4200b can insulate the circuit assembly 420 from the vibrational forces caused by the impaction sleeve 10, 20 transferring impaction forces to the tibial component 400. In some configurations, the at least one liner 4200a, 4200b can comprise one or more polymeric materials, such as polycarbonate. The at least one liner 4200a, 4200b can include a recess (not shown) configured to receive the identification tag 4202.

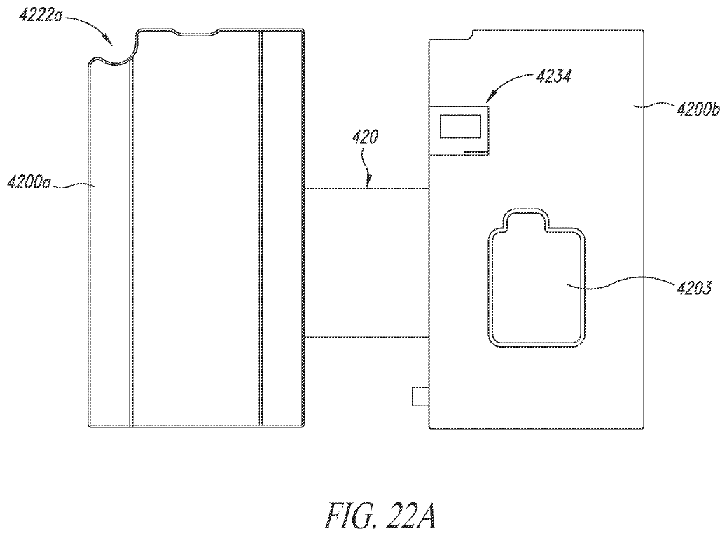

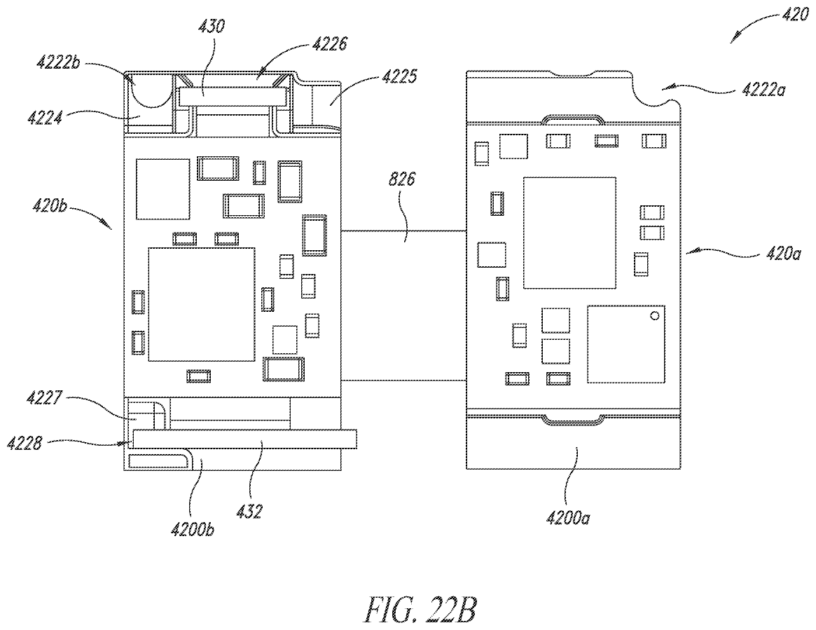

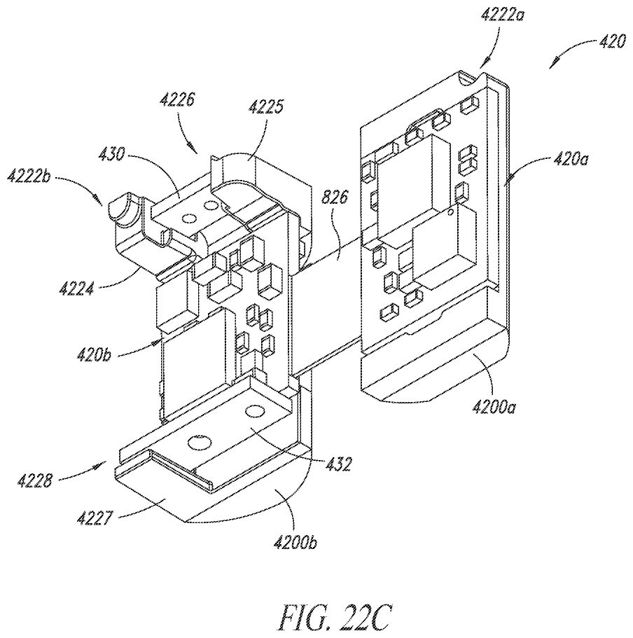

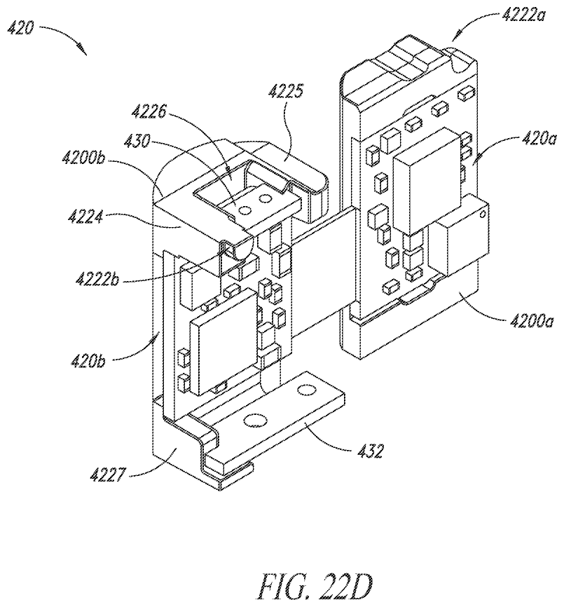

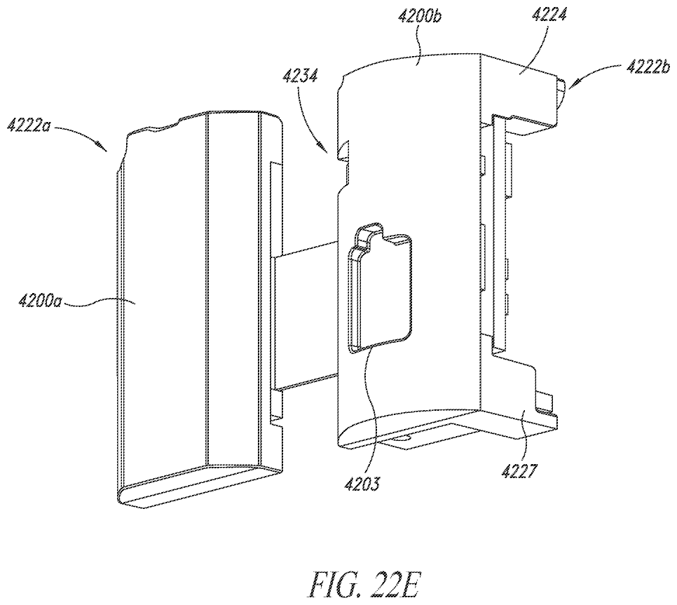

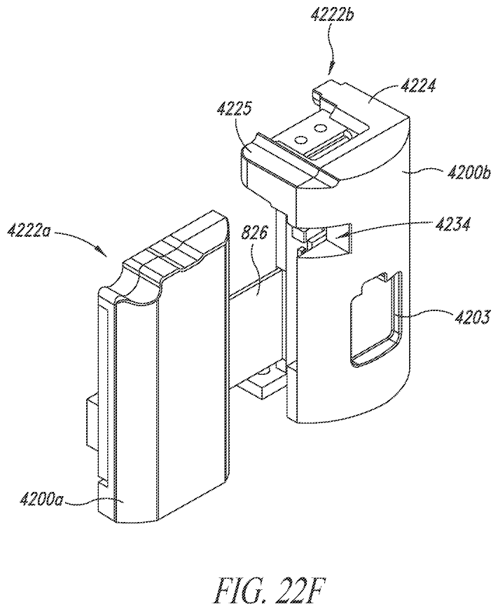

FIGS. 22A through 22F illustrate different views of the circuit assembly 420 with a different configuration of the first and second liners 4200a. FIGS. 22A and 22B illustrate a bottom view and a top view, respectively, of the circuit assembly 420 and the first and second liners 4200a, 4200b in a flat configuration, and FIGS. 22C-22F illustrate various perspective views of the circuit assembly 420 and the first and second liners 4200a, 4200b in the flat configuration.

As shown in FIGS. 22A and 22E-22F, the second liner 4200b can include a recess 4203 to receive the identification tag 4202 (not shown). In some configurations, the second liner 4200b can include one or more gaps 4234. For example, the one or more gaps 4234 can be positioned along the periphery of the second liner 4200b so that components of the circuit assembly 420 and align within the gap(s) 4234.

In some configurations, as shown in FIGS. 22B-22F, one or both of the liners 4200a, 4200b can include an alignment feature 4222a, 4222b. For example, the first liner 4200a can include an alignment feature 4222a. In some configurations, the alignment feature 4222a can include a cutout in the first liner 4200a. The second liner 4200b can include an alignment feature 4222b that can include a protrusion. The alignment feature 4222a of the first liner 4200a can engage with the alignment feature 4222b of the second liner 4200b when the circuit assembly 420 and the liners 4200a, 4200b are folded into the folded configuration. For example, the alignment feature 4222b can be received by and/or adhered to the alignment feature 4222a.

In some configurations, as shown in FIGS. 22B-22D, the first and second liners 4200a, 4200b can include additional alignment features 4226, 4228. For example, the second liner 4200b can include a first alignment feature 4226 in the form of a slot and a second alignment feature 4228 in the form of a slot. The first and second alignment features 4226, 4228 can be configured to receive portions of the circuit assembly 420. For example, the second portion 420b of the circuit assembly 420 can include one or more engagement features 430, 432. In some configurations, the engagement features 430, 432 can include a first engagement feature 430 and a second engagement feature 432. The first engagement features 430 may include includes an antenna terminal board. The second engagement feature 432 may include a battery terminal board 432. The first slot 4226 can be configured to receive the first engagement features 430. The second slot 4228 can be configured to receive the second engagement features 432. The first and/or second slots 4226, 4228 can be configured to define the orientation of the circuit assembly 420 within the liners 4200a, 4200b. For example, the first and/or second slots 4226, 4228 can align the second portion 420b of the circuit assembly 420 such that the longitudinal centerline of the second portion 420b of the circuit assembly 420 aligns with the longitudinal center line of the second liner 4200b.

The first and second slots 4226, 4228 can be formed by one or more protrusions 4224, 4225, 4227. For example, the first slot 4226 can be formed by two protrusions 4224, 4225. The two protrusions 4224, 4225 can include a first protrusion 4224 and a second protrusion 4225. In some configurations, the alignment feature 4222b can extend from at least one of the protrusions 4224, 4225. In the illustrated configuration, the alignment feature 4222b extends from the first protrusion 4224. The second slot 4228 can be at least partially formed by the third protrusion 4227.

In some configurations, each of the liners 4200a, 4200b can include an alignment feature comprising a rail (not shown). During manufacturing, the first portion 420a of the circuit assembly 420 can be adhered to a first rail (not shown) and the second portion 420b of the circuit assembly 420 can be adhered to a second rail (not shown).

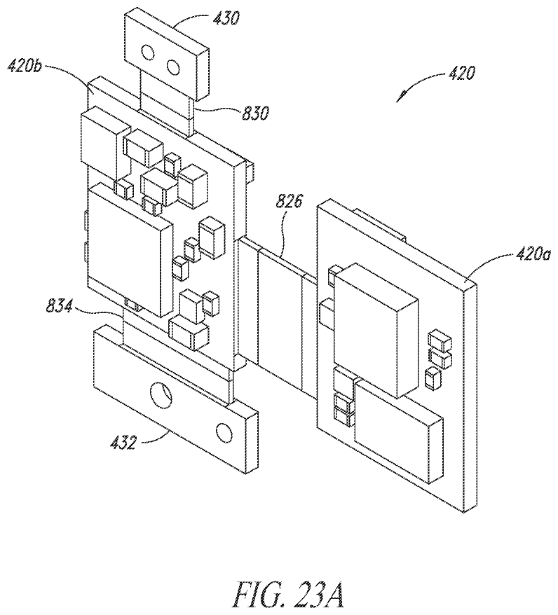

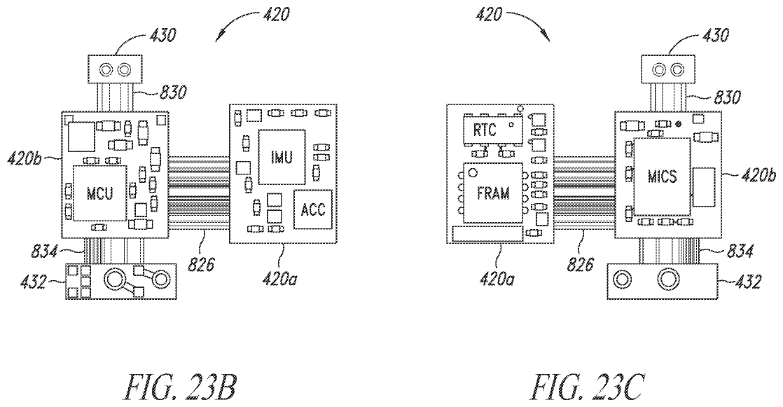

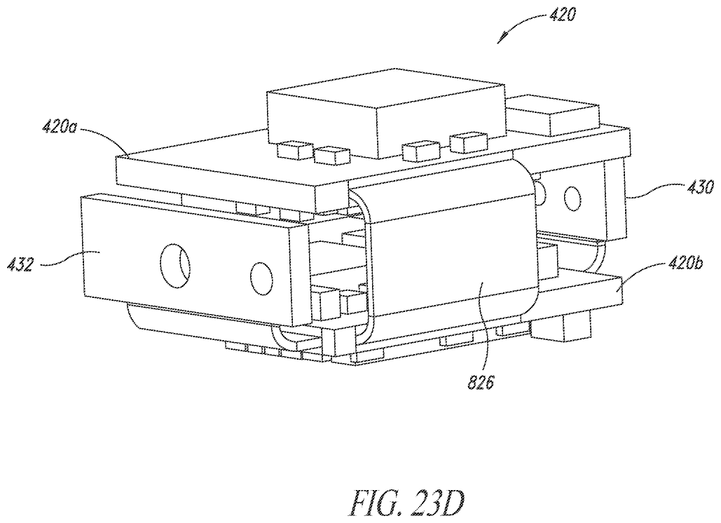

FIG. 23A illustrates a perspective view of the circuit assembly 420 in a flat configuration and FIG. 23B illustrates a perspective view of the circuit assembly 420 in a folded configuration. The circuit assembly 420 can include a first portion 420a and a second portion 420b. The first and second portions 420a, 420b can be folded from the flat configuration (FIG. 23A) to the folded configuration (FIG. 23B).

With reference to FIGS. 23B and 23C, each of the first portion 420a of circuit assembly 420 and the second portion 420b of the circuit assembly are circuit boards having various electronics components mounted thereon. The first portion 420a and the second portion 420b are connected to each other by a flex wire 826. The circuit assembly 420 also includes an antenna terminal board 430 that is connected to the first portion 420a by a flex wire 830, and a battery terminal board 432 that is connected to the second portion 420b by a flex wire 834. The flex wires 826, 830, 834 allows the circuit assembly 420 to be folded so that the two portions 420a, 420b are generally parallel to each other, and the two terminal boards 430, 432 are generally parallel to each other to form an open box structure.

FIGS. 24A through 24E illustrate different views of the circuit assembly 720 and/or the liners 7200a, 7200b, 7200c. The circuit assembly 720 and the liners 7200a, 7200b, 7200c can be the same as or similar to the embodiments of the circuit assembly 420 and the liners 4200a, 4200b illustrated in and described in relation to FIGS. 21A-23B. Reference numerals of the same or substantially the same features may share the same last two or three digits. The one or more liners 7200a, 7200b, 7200c can include a third liner 7200c. The third liner 7200c can be separate from the first and second liners 7200a, 7200b. In some configurations, the circuit assembly 720 may not be glued to the one or more liners 7200a, 7200b, 7200c. For example, the third liner 7200c can be positioned between the first and second portions 720a, 720b of the circuit assembly 720 such that the third liner 7200c can secure the first portion 720a of the circuit assembly 720 to the first liner 7200a and the second portion 720b of the circuit assembly 720 to the second liner 7200b. As shown in FIGS. 24C-24D, the first liner 7200a can include a recess 7203 configured to receive the identification tag 7202.

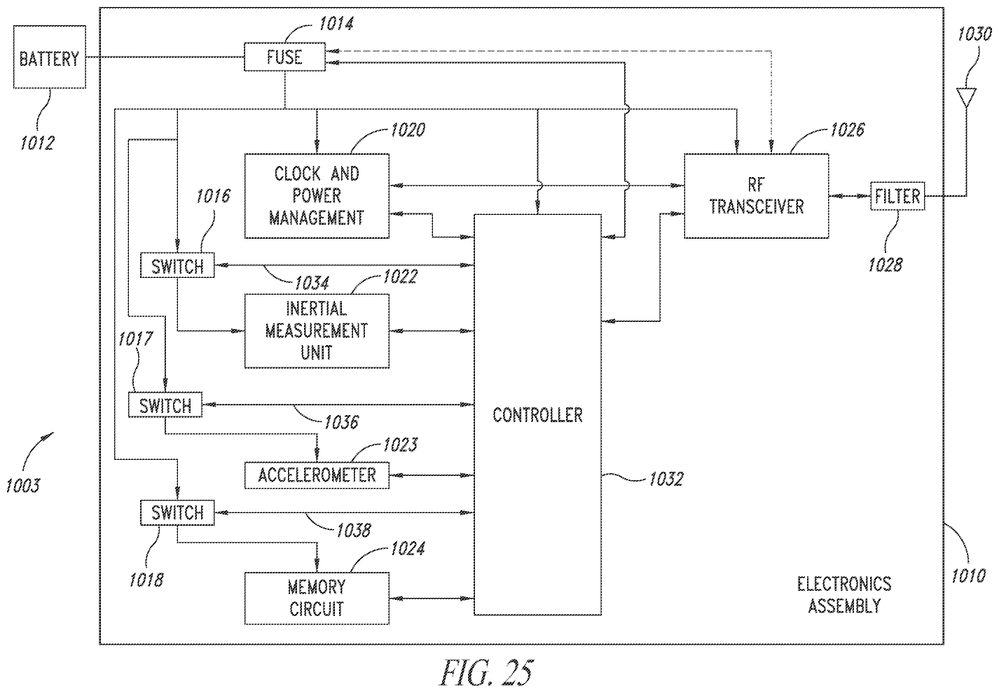

With reference to the block diagram of FIG. 25, an implantable reporting processor 1003 includes an electronics assembly 1010, a battery 1012 or other suitable implantable power source, and an antenna 1030. The electronics assembly 1010 comprises a circuit assembly that includes a fuse 1014, switches 1016, 1017, and 1018, a clock generator and clock and power management circuit 1020, an inertial measurement unit (IMU) 1022, a memory circuit 1024, a radio-frequency (RF) transceiver 1026, an RF filter 1028 and a controller 1032. The electronics assembly 1010 may also include an accelerometer 1023. The accelerometer 1023 may be a single axis or multi-axis accelerometer, and in one embodiment is a triaxial accelerometer. Examples of some or all of these components are described elsewhere in this application and in PCT Publication Nos. WO 2017/165717 and WO 2020/247890, which are incorporated by reference.

The battery 1012 can be any suitable battery, such as a Lithium Carbon Monofluoride (LiCFx) battery, or other storage cell configured to store energy for powering the electronics assembly 1010 for an expected lifetime (e.g., 5-25+ years) of the intelligent implant.

The fuse 1014 can be any suitable fuse (e.g., permanent) or circuit breaker (e.g., resettable) configured to prevent the battery 1012, or a current flowing from the battery, from injuring the patient and damaging the battery and one or more components of the electronics assembly 1010. For example, the fuse 1014 can be configured to prevent the battery 1012 from generating enough heat to burn the patient, to damage the electronics assembly 1010, to damage the battery, or to damage structural components of the kinematic implant.

The switch 1016 is configured to couple the battery 1012 to, or to uncouple the battery from, the IMU 1022 in response to a control signal 1034 from the controller 1032. For example, the controller 1032 may be configured to generate the control signal 1034 having an open state that causes the switch 1016 to open, and, therefore, to uncouple power from the IMU 1022, during a sleep mode or other low-power mode to save power, and, therefore, to extend the life of the battery 1012. Likewise, the controller 1032 also may be configured to generate the control signal 1034 having a closed state that causes the switch 1016 to close, and therefore, to couple power to the IMU 1022, upon “awakening” from a sleep mode or otherwise exiting another low-power mode. Such a low-power mode may be for only the IMU 1022 or for the IMU and one or more other components of the implantable reporting processor 1003.

The switch 1017 is configured to couple the battery 1012 to, or to uncouple the battery from, the accelerometer 1023 in response to a control signal 1036 from the controller 1032. For example, the controller 1032 may be configured to generate the control signal 1036 having an open state that causes the switch 1017 to open, and, therefore, to uncouple power from the accelerometer 1023, during a sleep mode to save power, and, therefore, to extend the life of the battery 1012. Likewise, the controller 1032 also may be configured to generate the control signal 1036 having a closed state that causes the switch 1017 to close, and therefore, to couple power to the accelerometer 1023, upon “awakening” from a sleep mode.

The switch 1018 is configured to couple the battery 1012 to, or to uncouple the battery from, the memory circuit 1024 in response to a control signal 1038 from the controller 1032. For example, the controller 1032 may be configured to generate the control signal 1038 having an open state that causes the switch 1018 to open, and, therefore, to uncouple power from the memory circuit 1024, during a sleep mode or other low-power mode to save power, and, therefore, to extend the life of the battery 1012. Likewise, the controller 1032 also may be configured to generate the control signal 1038 having a closed state that causes the switch 1018 to close, and therefore, to couple power to the memory circuit 1024, upon “awakening” from a sleep mode or otherwise exiting another low-power mode. Such a low-power mode may be for only the memory circuit 1024 or for the memory circuit and one or more other components of the electronics assembly 1010.

The clock circuit 1020 is configured to generate a clock signal for one or more of the other components of the electronics assembly 1010, and can be configured to generate periodic commands or other signals (e.g., interrupt requests) in response to which the controller 1032 causes one or more components of the implantable circuit to enter or to exit a sleep, or other low-power, mode. In some embodiments, the clock circuit 1020 is also configured to regulate the voltage from the battery 1012, and to provide a regulate power-supply voltage to some or all of the other components of the electronics assembly 1010. In these embodiments, the clock circuit 1020 may be referred to as a clock and power management circuit.

The IMU 1022 has a frame of reference with coordinate x, y, and z axes, and can be configured to measure, or to otherwise quantify, linear acceleration that the IMU experiences along each of the x, y, and z axes, and angular velocity (or rotational motion) that the IMU experiences about each of the x, y, and z axes. Such a configuration of the IMU 1022 is at least a six-axis configuration, because the IMU 1022 measures six unique quantities, aₓ(g), a_{y}(g), a_{z}(g), Ωₓ(dps), Ω_{y}(dps), and Ω_{z}(dps). Alternatively, the IMU 1022 can be configured in a nine-axis configuration, in which the IMU can use the earth magnetic field to compensate for, or to otherwise correct for, accumulated errors in aₓ(g), a_{y}(g), a_{z}(g), Ωₓ(dps), Ω_{y}(dps), and Ω_{z}(dps). But in an embodiment in which the IMU measures acceleration and angular velocity over only short bursts (e.g., 0.10-100 seconds(s)), for many applications accumulated error typically can be ignored without exceeding respective error tolerances.

The IMU 1022 can include a respective analog-to-digital converter (ADC) for each of the x, y, and z accelerometers and gyroscopes. Alternatively, the IMU 1022 can include a respective sample-and-hold circuit for each of the x, y, and z accelerometers and gyroscopes, and as few as one ADC that is shared by the accelerometers and gyroscopes. Including fewer than one ADC per accelerometer and gyroscope can decrease one or both of the size and circuit density of the IMU 1022, and can reduce the power consumption of the IMU. But because the IMU 1022 includes a respective sample-and-hold circuit for each accelerometer and each gyroscope, samples of the analog signals generated by the accelerometers and the gyroscopes can be taken at the same or different sample times, at the same or different sample rates, and with the same or different output data rates (ODR).

The accelerometer 1023 is configured to monitor acceleration in a low power state. The accelerometer 1023 may be a single axis or multi-axis accelerometer, and in one embodiment is a triaxial accelerometer. In the case of a triaxial configuration, the accelerometer 1023 can include a respective ADC for each of the x, y, and z accelerometers. Alternatively, the accelerometer 1023 can include a respective sample-and-hold circuit for each of the x, y, and z accelerometers, and as few as one ADC that is shared by the accelerometers. Including fewer than one ADC per accelerometer can decrease one or both of the size and circuit density of the accelerometer 1023, and can reduce the power consumption of the accelerometer 1023. Based on acceleration signals it senses, the accelerometer 1023 can detect motion events. For example, the accelerometer can be configured to detect simple motion events, such as footsteps or shoulder swings, and to count such detections. The accelerometer can be configured to detect significant motion, such as a walking motion or arm swinging motion. The accelerometer 1023 is configured to provide a wake-up signal to the controller 1032 when significant motion is detected.

The memory circuit 1024 can be any suitable nonvolatile memory circuit, such as EEPROM or FLASH memory, and can be configured to store data written by the controller 1032, and to provide data in response to a read command from the controller.

The RF transceiver 1026 can be a conventional transceiver that is configured to allow the controller 1032 (and optionally the fuse 1014) to communicate with a base station (not shown in FIG. 22) configured for use with the kinematic implantable device. For example, the RF transceiver 1026 can be any suitable type of transceiver (e.g., Bluetooth, Bluetooth Low Energy (BTLE), and WiFi®), can be configured for operation according to any suitable protocol (e.g., MICS, ISM, Bluetooth, Bluetooth Low Energy (BTLE), and WiFi®), and can be configured for operation in a frequency band that is within a range of 1 MHz-5.4 GHz, or that is within any other suitable range.

The RF filter 1028 can be any suitable bandpass filter, such as a surface acoustic wave (SAW) filter or a bulk acoustic wave (BAW) filter. In some embodiment, the RF filter 1028 includes multiple filters and other circuitry to enable dual-band communication. For example, the RF filter 1028 may include a bandpass filter for communications on a MICS channel, and a notch filter for communication on a different channel, such as a 2.45 GHz as described above with reference to FIG. 21.

The antenna 1030 can be any antenna suitable for the frequency band in which the RF transceiver 1026 generates signals for transmission by the antenna, and for the frequency band in which a base station generates signals for reception by the antenna. In some embodiments the antenna 1030 is configured as a flat ribbon loop antenna as described above with reference to FIGS. 20A-20E.

The controller 1032, which can be any suitable microcontroller or microprocessor, is configured to control the configuration and operation of one or more of the other components of the electronics assembly 1010. For example, the controller 1032 is configured to control the IMU 1022 to take measurements of movement of the implantable medical device with which the electronics assembly 1010 is associated, to quantify the quality of such measurements (e.g., is the measurement “good” or “bad”), to store measurement data generated by the IMU in the memory 1024, to generate messages that include the stored data as a payload, to packetize the messages, to provide the message packets to the RF transceiver 1026 for transmission to an external device, e.g. a base station. The controller 1032 may be configured to execute commands received from an external device via the antenna 1030, the RF filter 1028, and the RF transceiver 1026. For example, the controller 1032 can be configured to receive configuration data from a base station, and to provide the configuration data to the component of the electronics assembly 1010 to which the base station directed the configuration data. If the base station directed the configuration data to the controller 1032, then the controller is configured to configure itself in response to the configuration data. The controller 1032 may also be configured to execute data sampling by the IMU 1022 in accordance with one or more programmed sampling schedules, or in response to an on-demand data sampling command received from a base station. For example, as described later below, the implantable reporting processor 104 may be programmed to operate in accordance with a master sampling schedule and a periodic, e.g., daily, sampling schedule.

### Impaction Sleeve

The present disclosure provides a tool that may be used to bring two pieces of an intelligent implant together under force. More specifically, the tool is used to exert force on a first piece, where the first piece is adjacent to a second piece, and the second piece is held stationary. The force exerted on the tool is transmitted to the first piece, whereupon the first piece is pressed against the stationary second piece. The tool is intended for the situation where the first and second pieces have complementary mating surfaces, such that when the first and second pieces are forced against one another at the location of the mating surfaces, and under force generated through the tool of the present disclosure, the mating surfaces hold together, at least in part by frictional forces. In this way, two separate (first and second) pieces are combined to form a joined piece. The tool of the present disclosure is particularly advantageous in the situation where the first piece has both fragile and non-fragile regions, and the tool contacts the first piece at non-fragile regions only. In this way, a first piece having fragile regions can be pressed into a second piece, leaving the fragile regions unharmed. The tool is useful, for example, in assembling an alert implant of the present disclosure.

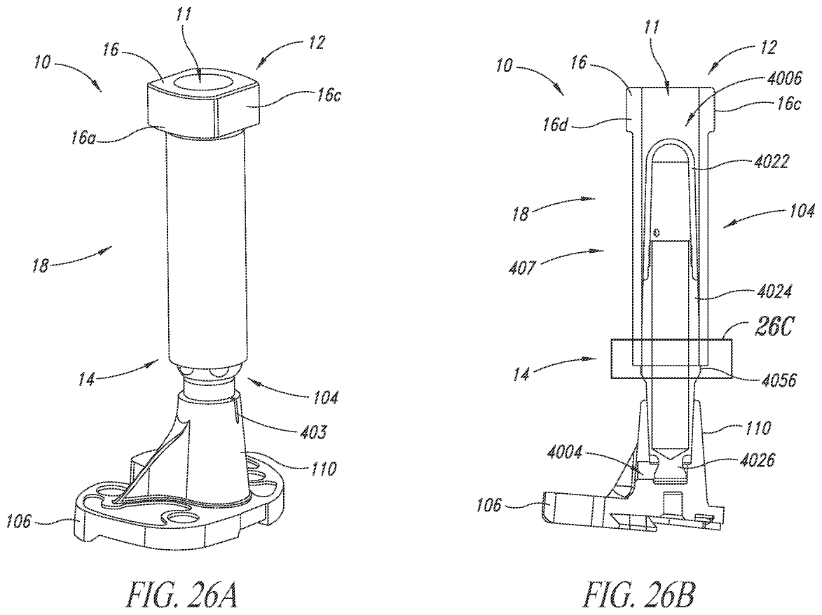

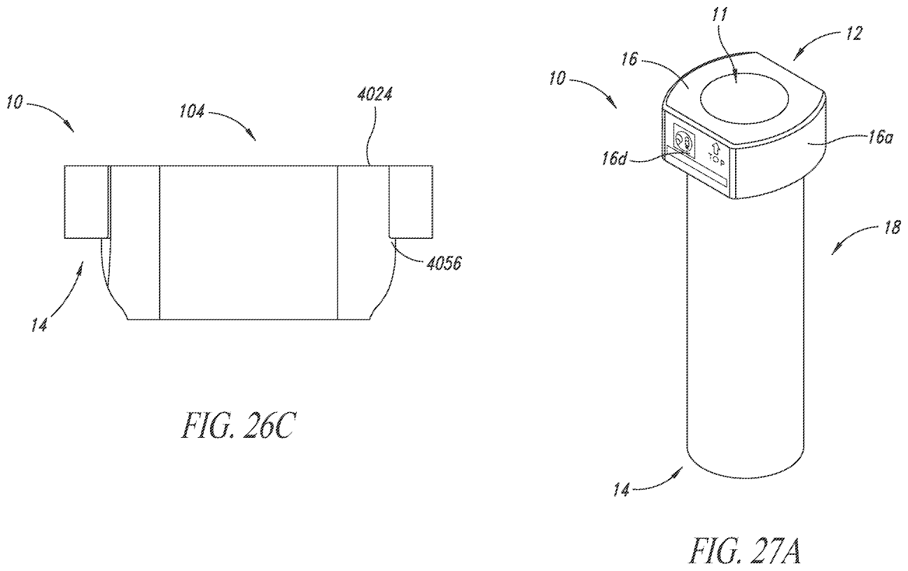

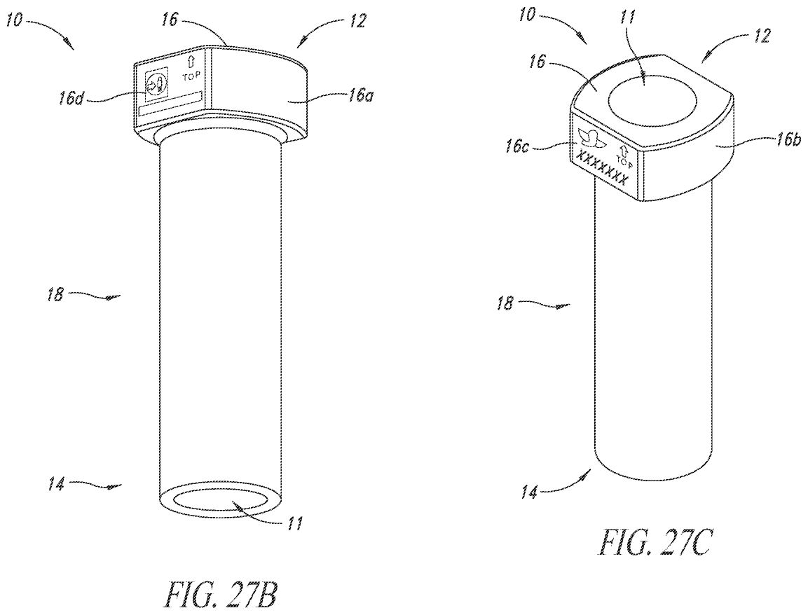

FIG. 26A illustrates a perspective view of a tool 10 or impaction sleeve coupled to an intelligent implant in the form of an implantable reporting processor 104 mechanically coupled to a tibial plate 106. FIGS. 26B-26C illustrate cross-sections of the tool 10 and the tibial plate 106. FIGS. 27A-27C illustrate different perspective views of the tool 10. In some configurations, the tool 10 can be an impaction sleeve 10 that transfers forces, such as impaction forces, from an impaction tool, such as a hammer, to the implantable reporting processor 104. For example, the impaction sleeve 10 can be used to couple the implantable reporting processor 104 with the tibial stem 110 of the tibial plate 106. In some configurations, the tibial plate 106 can include an alignment feature 403. For example, the illustrated configuration shows an alignment feature 403 that extends longitudinally from a distal edge of the tibial stem 110 of the tibial plate 106. The alignment feature 403 can be a line etched or otherwise formed onto an outer surface of the tibial stem 110 by a laser or other etching tool. The alignment feature 403 can be configured to engage with one or more corresponding alignment features of one or more of the other components of the intelligent implant. For example, during assembly, the alignment feature 403 of the tibial plate 106 can be aligned with an alignment feature 4027 of the implantable reporting processor 104, which is further described above in relation to FIG. 14A, such that the implantable reporting processor 104 is properly aligned with the tibial plate 106.

As shown in FIGS. 27A-27C, the impaction sleeve 10 can include a distal end 12 and a proximal end 14. The distal end 12 can include a distal head 16 that receives impaction forces from an impaction tool and an elongate portion 18 extending between the distal head 16 and the proximal end 14. In some configurations, an outer surface of the elongate portion 18 can include a constant width extending along a length of the elongate portion 18. In some configurations, the outer surface of the elongate portion 18 can have a varying width along the length of the elongate portion 18. The impaction sleeve 10 can include a channel 11. The channel 11 can extending through the distal end 12 and/or the proximal end 14. For example, the channel 11 can extend along a part of or the entire length of the impaction sleeve 10. In some configurations, the channel 11 can have a constant width along a length of the channel 11. In some configurations, the channel 11 can have a varying width along the length of the channel 11. The channel 11 can be sized and configured to receive a portion of the intelligent implant.

As shown in FIGS. 26A-26C, the channel 11 can receive at least the distal portion 407 of the implantable reporting processor 104. For example, the channel 11 can receive at least the cover 4022 and a body portion 4024 of the implantable reporting processor 104. In some configurations, the body portion 4024 of the implantable reporting processor 104 can include a shoulder 4056 closer to a proximal end 4004 of the implantable reporting processor 104 compared to a distal end 4006 of the implantable reporting processor 104. The proximal end 14 of the impaction sleeve 10 can engage or abut with the shoulder 4056 when the implantable reporting processor 104 is within the channel 11 such that the impaction sleeve 10 can transfer impaction forces to the shoulder 4056 to couple the coupling section 4026 of the implantable reporting processor 104 to the tibial plate 106. For example, the coupling section 4026 can engage with the tibial plate 402 via a press-fit engagement. In some configurations, the coupling section 4026 can engage with the tibial plate 106 via a threaded engagement, a snap-fit engagement, or other mechanical engagement.

The distal head 16 can have a width greater than a diameter of the elongate portion 18. In some configurations, the distal head 16 can have a rounded peripheral surface such that the distal head 16 can be substantially circular. In some configurations, the distal head 16 can have a peripheral surface with one or more of flat surfaces. The illustrated configuration shows the distal head 16 with two flat peripheral surfaces 16a, 16b and two rounded peripheral surfaces 16c, 16d. The two rounded peripheral surfaces 16c, 16d can be separated by each of the two flat peripheral surfaces 16a, 16b. The impaction sleeve 10 can comprise a metal material. For example, the metal material can be titanium.

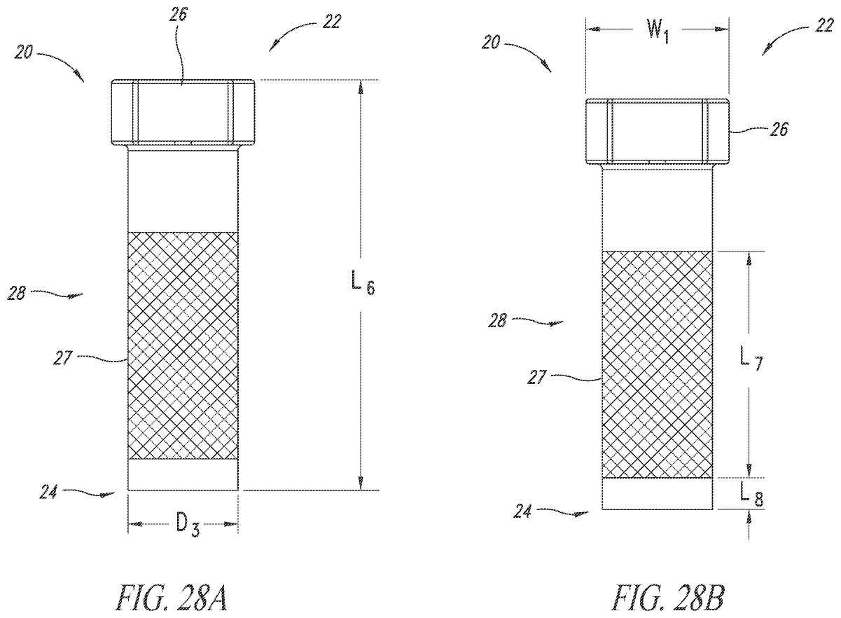

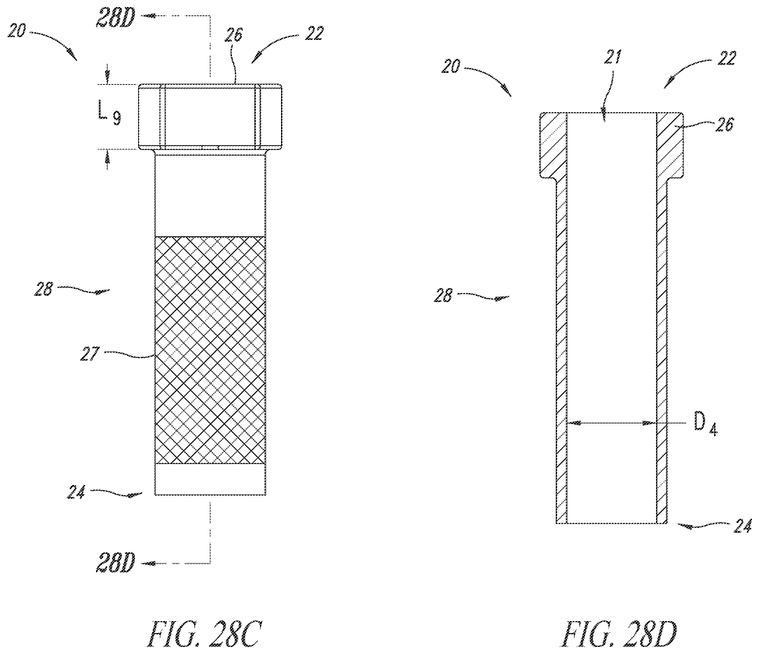

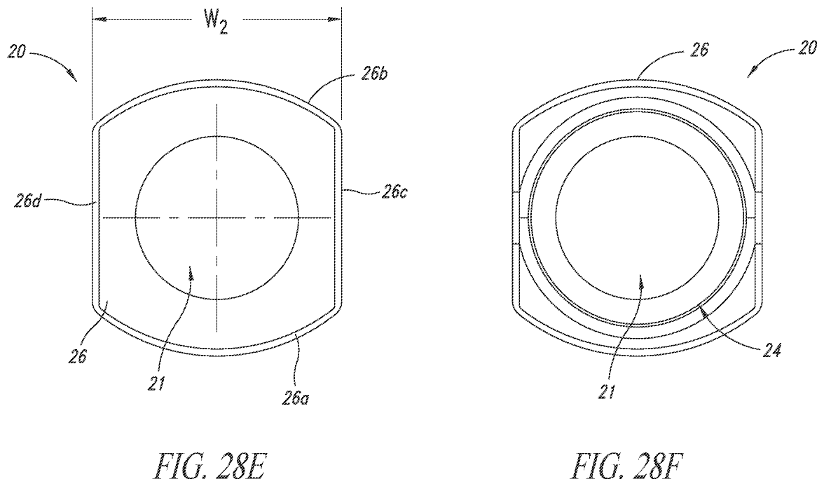

FIGS. 28A-28F illustrate different views of another embodiment of a tool 20 or impaction sleeve. The impaction sleeve 20 can be the same as or similar to the embodiments of the impaction sleeve 10 illustrated in and described in relation to FIGS. 27A-27C. Reference numerals of the same or substantially the same features may share the same last digit.

FIGS. 28A-28C illustrate side views of the impaction sleeve 20. As shown in FIG. 28A, the impaction sleeve 20 can have a total length Le of between about 30 mm and about 100 mm, about 40 mm and about 90 mm, about 50 mm and about 80 mm, about 60 mm and about 70 mm, or about 63.5 mm. The elongate portion 28 of the impaction sleeve 20 can have a diameter D3 of between about 5 mm and about 30 mm, about 10 mm and about 20 mm, or about 17.15 mm.

As shown in FIG. 28B, the distal head 26 of the impaction sleeve 20 can have a maximum width W₁ of between about 5 mm and about 50 mm, about 10 mm and about 40 mm, about 20 mm and about 30 mm, or about 22 mm. As shown in FIG. 28C, the distal head 26 of the impaction sleeve 20 can have a length L₉ of between about 5 mm and about 20 mm, or about 10 mm.

As shown in FIGS. 28A-28C, an outer surface of the impaction sleeve 20 can be modified to form a gripping surface 27. Advantageously, the gripping surface 27 allows the impaction sleeve 20 to be easier to handle, especially during a procedure when the clinician is wearing gloves. The gripping surface 27 can extend along the elongate portion 28 of the impaction sleeve. For example, as shown in FIG. 28B, the gripping surface 27 can have a length L₇ of between about 30 mm and about 100 mm, about 40 mm and about 90 mm, about 50 mm and about 80 mm, about 60 mm and about 70 mm, or about 63.5 mm. A proximal end of the gripping surface 27 can be spaced from the proximal end 24 of the impaction sleeve 20. For example, a distance La between the gripping surface 27 and the proximal end 24 can be between about 1 mm and about 10 mm, or about 5 mm.

FIG. 28D illustrates a cross-sectional view of the impaction sleeve 20. The channel 21 can have a diameter D₄ of between about 5 mm and about 30 mm, about 10 mm and about 20 mm, or about 13 mm. FIG. 28E illustrates a top view of the impaction sleeve 20 and FIG. 28F illustrates a bottom view of the impaction sleeve 20. As shown in FIG. 28E, the distal head 26 can have a minimum width W₂ of between about 5 mm and about 40 mm, about 10 mm and about 30 mm, or about 20 mm.

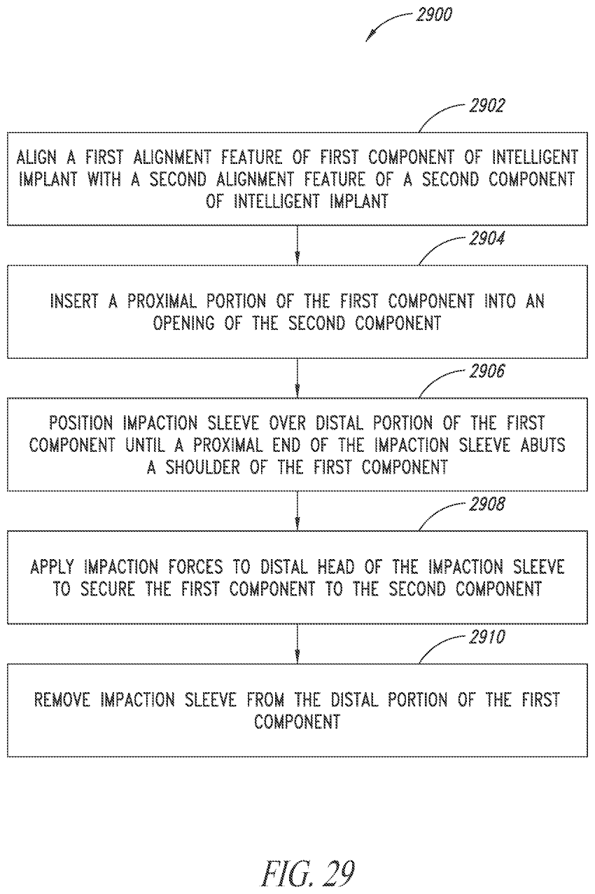

FIG. 29 is a flowchart of a method of assembling a first component and a second component of an intelligent implant. The method can be performed using the tool 10, e.g., impaction sleeve, of FIGS. 26A-28E. The first component of the intelligent implant may be an implantable reporting processor. The second component of the intelligent implant may be a tibial plate of a knee replacement system, a humeral stem of a shoulder replacement system, a femoral stem of a knee replacement system, or any other component of a joint replacement system.

At block 2902, a first alignment feature of the first component is aligned with a second alignment feature of the second component.

At block 2904, a proximal portion of a first component of an intelligent implant is inserted into an opening of a second component of the intelligent implant.

At block 2906, an impaction sleeve 10 is positioned over a distal portion of the first component until a proximal end of the impaction sleeve abuts a shoulder of the first component.

At block 2908, impaction forces are applied to a distal head of the impaction sleeve to secure the first component to the second component. The impaction forces are transferred from the impaction sleeve to the shoulder of the first component.

At block 2910, the impaction sleeve is removed from the distal portion of the first component.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof such as “a configuration” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include,” as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

In the foregoing description, certain specific details are set forth to provide a thorough understanding of various disclosed embodiments. However, one skilled in the relevant art will recognize that embodiments may be practiced without one or more of these specific details, or with other methods, components, materials, etc. In other instances, well-known structures associated with electronic and computing systems including client and server computing systems, as well as networks have not been shown or described in detail to avoid unnecessarily obscuring descriptions of the embodiments.

Although any methods and materials similar or equivalent to those described herein can also be used in the practice or testing of the present invention, a limited number of the exemplary methods and materials are described herein. Generally, unless otherwise indicated, the materials for making the invention and/or its components may be selected from appropriate materials such as metal, metallic alloys, ceramics, plastics, etc.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the term “about” means ±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet are incorporated herein by reference, in their entireties. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An implantable reporting processor comprising:
a casing configured to house at least a portion of an electronics assembly, the casing comprising a distal facing wall and an externally threaded portion positioned distally from the distal facing wall, the externally threaded portion separated from the distal facing wall by an externally smooth surface; and
a cover configured to house an antenna electrically couple to the electronics assembly, wherein the cover comprises an internally threaded portion spaced apart from a proximal edge of the cover by an internally smooth surface, wherein the externally threaded portion of the casing is configured to engage with the internally threaded portion of the cover.

2. The implantable reporting processor of claim 1, wherein at least a portion of the casing comprises a metal material.

3. The implantable reporting processor of claim 1, wherein the cover comprises a plastic material.

4. The implantable reporting processor of claim 1, wherein the cover comprises a proximally facing edge, wherein the distal facing wall is configured to abut the proximally facing edge of the cover.

5. The implantable reporting processor of claim 4, wherein other than the externally and internally threaded portions, the cover only contacts the casing at the proximally facing edge of the cover.

6. The implantable reporting processor of claim 1, wherein the internally smooth surface of the cover tapers from the proximal edge to the internally threaded portion.

7. The implantable reporting processor of claim 1, wherein the externally smooth surface of the casing is tapered.

8. The implantable reporting processor of claim 1, wherein the internally smooth surface is spaced apart from the externally smooth surface when the cover is coupled to the casing.

9. The implantable reporting processor of claim 1, wherein the distal facing wall is configured to abut a proximally facing edge of the cover so that an outer surface of the casing is flush with an outer surface of the cover.

10. The implantable reporting processor of claim 1, wherein the externally threaded portion comprises a reduced diameter compared to a portion of the casing that is proximal of and adjacent to the distal facing wall.

11. An implantable reporting processor comprising:
a casing comprising an externally threaded portion positioned distally from an externally smooth surface and a distal facing wall adjacent the externally smooth surface; and
a cover comprising an internally threaded portion spaced apart from an edge of the cover by an internally smooth surface, wherein the externally threaded portion of the casing is configured to engage with the internally threaded portion of the cover, the edge is configured to abut the distal facing wall of the casing, other than the externally and internally threaded portions, the cover only contacts the casing at the edge of the cover, the internally smooth surface of the cover tapers from the edge to the internally threaded portion, the externally smooth surface of the casing is tapered, and the internally smooth surface is spaced apart from the externally smooth surface when the cover is coupled to the casing.

12. The implantable reporting processor of claim 11, wherein at least a portion of the casing comprises a metal material.

13. The implantable reporting processor of claim 11, wherein the cover comprises a plastic material.

14. The implantable reporting processor of claim 11, wherein an outer surface of the casing is flush with an outer surface of the cover when the cover is coupled to the casing.

15. The implantable reporting processor of claim 11, wherein the externally threaded portion comprises a reduced diameter compared to a portion of the casing that is proximal of and adjacent to the distal facing wall.

16. The implantable reporting processor of claim 11, further comprising an electronics assembly and an antenna, wherein the casing is configured to house the electronics assembly.

17. The implantable reporting processor of claim 16, wherein the cover is configured to house the antenna.

18. An intelligent implant comprising:
an implantable reporting processor configured in accordance with one or more of claims 1-17; and
a component of a prothesis system having a receptacle configured to receive a portion of the implantable reporting processor and to mechanically couple with the implantable reporting processor.

19. The intelligent implant of claim 18, wherein the component of a prothesis system comprises one of a tibial component or femoral component of a knee prosthesis system, a humeral component of a shoulder prosthesis system, and a femoral component of a hip prosthesis system.

20-174. (canceled)
